(19) United States
(12) Patent Application Publication
(10) Pub. No.: US 2024/0262784 A1
(43) Pub. Date: Aug. 8, 2024

(54) METHOD FOR PRODUCING ASPARTIC ACID

(71) Applicants: Green Earth Institute Co., Ltd., Tokyo (JP); DIC Corporation, Tokyo (JP)

(72) Inventors: Satomi MIURA, Chiba (JP); Toru NAKAYASHIKI, Chiba (JP); Yusho USAMI, Chiba (JP)

(73) Assignees: Green Earth Institute Co., Ltd., Tokyo (JP); DIC Corporation, Tokyo (JP)

(21) Appl. No.: 18/566,108

(22) PCT Filed: Jun. 27, 2022
(86) PCT No.: PCT/JP2022/025631

§ 371 Date: Dec. 1, 2023

(30) Foreign Application Priority Data
Jun. 29, 2021 (JP) 2021-107301

(51) Int. Cl. C07C 227/42; C12P 13/20

(52) U.S. Cl. CPC C07C 227/42; C12P 13/20

(57) ABSTRACT

The present disclosure pertains to a method for producing aspartic acid whereby impurities can be reduced or eliminated even when a crude product contaminated with a considerable amount of impurities such as amino acids, organic substances, colorants, and inorganic salts is used as a starting material. The method comprises: (q) preparing a slurry of a crystalline fraction (X) containing β-type crystals of aspartic acid and at least one impurity; and (r) heating the slurry to convert the β-type crystals of aspartic acid to α-type crystals and obtain a crystalline fraction (Y) containing aspartic acid in the α-type crystals.

### TECHNICAL FIELD

The present invention pertains to a method for producing aspartic acid. In detail, the present invention pertains to a method for producing aspartic acid having an α-type crystal form.

### BACKGROUND ART

A variety of amino acids are the configuration units of proteins configuring living bodies and substances capable of exhibiting a variety of biological or chemical functions and are thus in use in a wide range of applications as raw materials of food raw materials, medicines, chemical materials, cosmetics and the like. Particularly, aspartic acid or glutamic acid, which is known as an acidic amino acid, is used as food raw materials such as sweeteners and flavor enhancers and, in recent years, polyaspartic acid or polyglutamic acid obtained by polymerizing aspartic acid or glutamic acid also has been gaining attention as an environmental-friendly functional material retaining biodegradable, water-absorbing or other functions. With such a background, a variety of production methods or purification techniques for aspartic acid or glutamic acid have been developed.

For example, Patent Literature 1 discloses a method for obtaining a purified optically active β-type glutamic acid crystal, in which optically active glutamic acid crude crystals containing α-type glutamic acid crystals and crystal mud composed of an aqueous solvent are left to stand or stirred in a temperature range of 50° C. or higher and 120° C. or lower and then glutamic acid crystals are separated and obtained. In Patent Literature 1, not only the fact that the total amount of a treatment system is small and the amount of energy or effort necessary for treatments can also be reduced compared with conventional methods but also the fact that the addition of an acid or an alkali is not required and thus a disadvantageous phenomenon, such as the contamination of products with sodium chloride or the like or optically active racemization, can be suppressed are mentioned as the advantages of the same method.

Furthermore, Patent Literature 2 discloses a method for purifying aspartic acid, in which aspartic acid crystals containing at least Cl⁻ are purified in an aqueous solution under suspension at a temperature of 50° C. or higher. In Patent Literature 2, it is suggested that, in crude crystals of aspartic acid that is produced by a fermentation method, an enzyme method and a chemical synthesis method, other impurities such as amino acids, colorants, and inorganic salts are contained, and thus, according to the purification method described in the same literature, it is possible to obtain a high-purity aspartic acid crystal from which impurities such as Cl⁻ have been further reduced or eliminated compared with conventional methods.

Furthermore, Patent Literature 3 discloses a method for crystallizing aspartic acid by mixing an ammonium aspartate aqueous solution and sulfuric acid or hydrochloric acid, in which a specific amount of malic acid is made to coexist in a crystallization system. In the method according to Patent Literature 3, it is suggested that an objective is to crystallize conventional columnar crystals, a washing effect in a washing step is improved, and high-purity aspartic acid columnar crystals are obtained.

Recently, in the industrial production of a variety of amino acids including aspartic acid or glutamic acid, an enzyme method or an extraction method has been thus far generally used. For example, in the production of aspartic acid by the enzyme method, a method in which fumaric acid and ammonia are used as raw materials and aspartic acid is synthesized using a reversible reaction by aspartase has been employed, and, as a method for separating and purifying the synthesized aspartic acid, a separation and purification method in which a variety of treatments such as adsorption by activated carbon, filtration, isoelectric crystallization, cooling, crystal separation, drying and immobilization using a carrageenan carrier are appropriately combined together has been employed. On the other hand, in the production by the extraction method, a method in which a predetermined protein is subjected to a hydrolysis reaction by hydrochloric acid or the like to be degraded into amino acid units and a desired amino acid is isolated/purified has been employed. At this time, for the isolation/purification of a desired amino acid, a method for isolating/purifying a target amino acid by combining a variety of separation methods such as ion exchange chromatography and fractional crystallization based on a difference in isoelectric point, adsorption properties, solubility or the like in each substance is employed. Furthermore, in recent years, a fermentation method in which a microbe is used also has been becoming usual. For example, a method in which Pantoea ananatis imparted with an L-glutamic acid production capability is cultured in a culture medium adjusted to a pH condition, under which L-glutamic acid is deposited, and the crystals of L-glutamic acid are generated and accumulated while being deposited in the culture medium is known (Patent Literature 4). In this method for producing L-glutamic acid, when the L-glutamic acid concentration in the culture medium is lower than a concentration at which natural crystallization occurs, α-type crystals of L-glutamic acid are deposited by making L-lysine be present in the culture medium.

### CITATION LIST

### Patent Literature

### Patent Literature 1

Japanese Patent Publication No. S45-4730

### Patent Literature 2

Japanese Patent Laid-Open No. S63-233958

### Patent Literature 3

Japanese Patent Laid-Open No. H8-217733

### Patent Literature 4

Pamphlet of International Publication WO 2004/099426

### SUMMARY OF INVENTION

### Technical Problem

In the case of producing aspartic acid through a bioprocess such as a fermentation method in which a microbe is used, which is different from an enzyme method and an extraction method, which are generally used at the moment, a crude product to be obtained is contaminated with, aside from intended aspartic acid, a considerable amount of other impurities such as amino acids, organic substances, colorants, and inorganic salts derived from the microbe, a culture medium component or the like. The present inventors found that it becomes a new objective to establish a technique for separating and purifying or producing aspartic acid that enables the effective reduction or elimination of impurities in such crude products contaminated with a considerable amount of impurities.

### Solution to Problem

In order to attain this objective, the present inventors intensively examined a variety of conditions for separating and purifying aspartic acid from a crude product sample that is derived from a culture of a microbe and contains a variety of impurities and consequently found that a variety of impurities are effectively reduced or eliminated and high-purity aspartic acid can be produced in an α-type crystal form by a process of preparing β-type crystals of aspartic acid separated from the crude product sample in a slurry form, heating this slurry, converting the β-type crystals to α-type crystals and obtaining a crystalline fraction containing the α-type crystals. The present invention is an invention completed by such a finding.

According to aspects of the present invention, the following methods for producing aspartic acid are provided.
[1] A method for producing aspartic acid, including:
   (q) preparing a slurry of a crystalline fraction (X) containing β-type crystals of aspartic acid and at least one impurity; and
   (r) heating the slurry to convert the β-type crystals of aspartic acid to α-type crystals and then obtain a crystalline fraction (Y) containing aspartic acid in the α-type crystals.
[2] The method according to [1], in which, in the step (r), the slurry is heated in a temperature range of 30° C. to 190° C., preferably 60° C. to 190° C., to convert the β-type crystals of aspartic acid to α-type crystals.
[3] The method according to [1] or [2], in which, in the step (r), the slurry is heated in a temperature range of 65° C. to 150° C. to convert the β-type crystals of aspartic acid to α-type crystals.
[4] The method according to any one of [1] to [3], further including:
   (p) in a solution (S) containing aspartic acid or a salt thereof and at least one impurity, adjusting a pH of the solution (S) to a predetermined pH value in an acidic region to generate β-type crystals of aspartic acid and then separate a fraction containing the β-type crystals from the solution (S),
   in which, in the step (q), a slurry of the crystalline fraction (X) is prepared using the fraction containing the β-type crystals.
[5] The method according to [4], in which, in the step (p), the pH of the solution (S) is adjusted to a predetermined value within a range of 0.50 to 6.95 to generate the β-type crystals of aspartic acid.
[6] The method according to [4] or [5], in which, in the step (p), the pH of the solution (S) is adjusted to a predetermined value within a range of 1.50 to 4.50 to generate the β-type crystals of aspartic acid.
[7] The method according to any one of [4] to [6], in which the solution (S) that is subjected to the step (p) contains a seed crystal.
[8] The method according to [7], in which the seed crystal contains β-type crystals of aspartic acid.
[9] The method according to any one of [4] to [8], in which the solution (S) that is subjected to the step (p) is a culture obtained by culturing or reacting a microbe in a culture medium, a clear liquid separated from the culture or a concentrate thereof.
[10] The method according to any one of [4] to [9], in which the solution (S) that is subjected to the step (p) is a solution containing the aspartic acid or a salt thereof at a concentration of 0.1 to 5.0 M.
[11] The method according to any one of [4] to [10], in which the solution (S) that is subjected to the step (p) contains, as the impurity, at least one selected from the group consisting of amino acids other than aspartic acid, organic acids, and salts thereof.
[12] The method according to any one of [4] to [11], in which the solution (S) that is subjected to the step (p) contains, as the impurity, at least
   i) at least one selected from the group consisting of glutamic acid, alanine, valine, and a salt thereof, and
   ii) at least one selected from the group consisting of pyruvic acid, malic acid, acetic acid, succinic acid, fumaric acid, and a salt thereof.
[13] The method according to any one of [4] to [12], in which the pH of the solution (S) that is subjected to the step (p) is within a range of 6.00 to 8.00.
[14] The method according to [13], in which, in the step (p), an acid is added to the solution (S) to adjust the pH of the solution (S) to a predetermined value within a range of 1.00 to 6.85 and generate the β-type crystals of aspartic acid.
[15] The method according to any one of [4] to [15], in which,
   in the step (p), after the β-type crystals of aspartic acid are generated in the solution (S), the fraction containing the β-type crystals is separated from the solution (S) by a solid-liquid separation method,
   in the step (q), the slurry of the crystalline fraction (X) is prepared using the fraction containing the β-type crystals separated in the step (p), and,
   in the step (r), the slurry is heated, the β-type crystals of aspartic acid are converted to α-type crystals, and then the crystalline fraction (Y) is separated from the slurry of the crystalline fraction (X) by the solid-liquid separation method.
[16] The method according to [15], in which,
   in the step (p), after a crystalline fraction containing the β-type crystals is separated from the solution (S) by the solid-liquid separation method, the separated crystalline fraction is washed with a solvent once or more and, furthermore, dried, in the step (q), the slurry of the crystalline fraction (X) is prepared using the dried crystalline fraction, and,
   in the step (r), after the crystalline fraction (Y) is separated from the slurry of the crystalline fraction (X) by the solid-liquid separation method, the separated crystalline fraction (Y) is washed with a solvent once or more and, furthermore, dried.

### Advantageous Effects of Invention

According to an embodiment of the present invention, even when a crude product contaminated with a considerable amount of impurities such as amino acids other than aspartic acid, organic acids, proteins, organic substances containing a saccharide, and inorganic salts or the like is used as a starting material, it is possible to produce high-purity aspartic acid, from which these impurities have been reduced or eliminated, in an α-type crystal form. Furthermore, according to a specific embodiment of the present invention, it is possible to produce high-purity α-type crystals of aspartic acid, from which, particularly, colorants have been reduced or eliminated.

### BRIEF DESCRIPTION OF DRAWINGS

FIG. 1 is a view schematically showing an example of each step that can be employed in a method according to the present invention.

FIG. 2A is a view showing a result of an amino acid/organic acid analysis of a filtrate (A) in Test Example 1.

FIG. 2B is a view showing a result of an amino acid/organic acid analysis of a crude crystal (B) in Test Example 1.

FIG. 2C is a view showing a result of an amino acid/organic acid analysis of a crystal (C) in Test Example 1.

FIG. 3A is a view showing the proportions of a variety of impurities eliminated from each sample through an isoelectric crystallization step in Test Example 1.

FIG. 3B is a view showing the proportions of a variety of impurities eliminated from each sample through a thermal re-slurry treatment step in Test Example 1.

FIG. 3C is a view showing the proportions of a variety of impurities eliminated from each sample through both of isoelectric crystallization and a thermal re-slurry treatment in Test Example 1.

FIG. 4A is a view showing a photograph of the appearances of crystal samples obtained in Test Example 1.

FIG. 4B is a view showing a micrograph of the crystal samples obtained in Test Example 1.

FIG. 4C is a view showing a micrograph of the crystal samples obtained in Test Example 1.

FIG. 4D is a view showing a micrograph of the crystal samples obtained in Test Example 1.

FIG. 5A is a view showing an X-ray diffraction chart of a crude crystal (B) in Test Example 1.

FIG. 5B is a view showing an X-ray diffraction chart of a crystal (C) in Test Example 1.

FIG. 6 is a view showing the analysis results of a variety of amino acids of each sample in Test Example 2.

FIG. 7 is a view showing the analysis results of a variety of organic acids of each sample in Test Example 2.

FIG. 8A is a view showing the aspartic acid collection rate in each sample in Test Example 2.

FIG. 8B is a view showing the residual rates of a variety of impurities in a crude crystal (B) in Test Example 2.

FIG. 9 is a view showing the residual rates of a variety of impurities in each crystal sample in Test Example 2.

FIG. 10 is a view showing the results of an evaluation test of a final product in Test Example 2.

FIG. 11 is a view showing photographs of the appearance of a no-seed crystal-added sample captured at the time of isoelectric crystallization in Test Example 3.

FIG. 12 is a view showing the residual rates of aspartic acid and other amino acids in each crystal sample in Test Example 3.

FIG. 13 is a view showing the residual rates of a variety of organic acids in each crystal sample in Test Example 3.

FIG. 14 is a view showing the analysis results of a variety of amino acids in each sample in Test Example 5.

FIG. 15 is a view showing the analysis results of a variety of organic acids and dihydroxyacetone (DHA) in each sample in Test Example 5.

FIG. 16A is a view showing aspartic acid collection rates (residual rates) in each sample according to a crude crystal (B) and a crystal (C) obtained in Test Example 5.

FIG. 16B is a view showing the residual rate of each impurity in the crude crystal (B) obtained in Test Example 5.

FIG. 17 is a view showing the residual rate of each impurity in each sample according to each crystal (C) obtained in Test Example 5.

FIG. 18 is a view showing the results of microscopic observation of a crystal conversion in each sample due to a thermal re-slurry treatment in Test Example 5.

### DESCRIPTION OF EMBODIMENTS

As described above, according to aspects of the present invention,

provided is a method for producing aspartic acid, including:

(q) preparing a slurry of a crystalline fraction (X) containing β-type crystals of aspartic acid and at least one impurity; and

(r) heating the slurry to convert the β-type crystals of aspartic acid to α-type crystals and then obtain a crystalline fraction (Y) containing aspartic acid in the α-type crystals.

Furthermore, in a specific embodiment, the method according to the present invention further includes, before the step (q):

(p) in a solution (S) containing aspartic acid or a salt thereof and at least one impurity, adjusting the pH of the solution (S) to a predetermined pH value in an acidic region to generate β-type crystals of aspartic acid and then separate a fraction containing the β-type crystals from the solution (S).

Here, in the subsequent step (q), a slurry of the crystalline fraction (X) is prepared using the fraction containing the β-type crystals.

Hereinafter, each term in the present invention will be described, and the steps (p), (q), and (r) will be sequentially described regarding an embodiment in which the method according to the present invention can be employed.

### Description of Terms

In the present invention, “aspartic acid” and “salt of aspartic acid” simply need to be interpreted literally. In the present invention, aspartic acid can be an L body or a D body, which is abundant in nature, or a mixture thereof. Additionally, a salt of aspartic acid that can be employed in the present invention is not particularly limited, and examples thereof include an ammonium salt, a sodium salt, a potassium salt, a calcium salt, and the like. Furthermore, in the present invention, aspartic acid or a salt thereof may include a form of an anhydride or a hydrate (for example, a monohydrate or a dihydrate) of aspartic acid or a salt thereof. In the case of producing a salt of aspartic acid by a bioprocess such as a microbe fermentation method, normally, the solution (S) may mainly contain L-aspartic acid or a salt thereof. Here, unless particularly otherwise described, terms referred to as “aspartic acid” and “salt thereof (salt of aspartic acid)” in the present invention simply need to be interpreted literally and are not limited to specific configurations unless particularly otherwise described.

In the present invention, “impurity” refers to a variety of substances other than aspartic acid and a salt thereof, which is considered as a production subject, and refers to a variety of substances reduced or eliminated from a crude product to separate and purify aspartic acid. Examples of “impurity” include a variety of amino acids other than aspartic acid (for example, glycine, alanine, serine, threonine, asparagine, glutamine, lysine, arginine, histidine, valine, leucine, isoleucine, tyrosine, phenylalanine, tryptophan, proline, methionine, and cysteine) and salts thereof, other organic acids (for example, pyruvic acid, malic acid, acetic acid, succinic acid, and fumaric acid) and salts thereof, glycoproteins such as proteins, peptides, carbohydrates, saccharides, and peptidoglycan, inorganic salts, and inorganic ions such as SO₄²⁻ or Cl⁻.

A subject that is produced by the method according to the present invention is aspartic acid in an α-type crystal form, but forms that are included in starting materials or crude products that are subjected to the method may be not only aspartic acid but also a salt of aspartic acid, and, in this case, it is needless to say that a salt of aspartic acid that can be converted to aspartic acid in α-type crystals in the end is not an impurity.

In the present invention, each of the terms “α-type crystals” and “β-type crystals” means crystal polymorphism that aspartic acid may have, which is as known to persons skilled in the art, and needs to be interpreted literally. Specifically, α-type crystals are observed as plate-like crystals when observed with a microscope or the like and have peaks at diffraction angles near 21.65° and 23.7° in an X-ray diffraction pattern when analyzed by X-ray diffraction. On the other hand, β-type crystals are observed as fine columnar crystals when observed with a microscope or the like and have peaks at diffraction angles near 18.8°, 19.7°, and 25.0° in an X-ray diffraction pattern when analyzed by X-ray diffraction.

### Step (p)

The solution (S) that is subjected to the step (p) may contain, aside from aspartic acid or a salt thereof, at least one of the above-described substances as an impurity. According to a specific embodiment, as shown in examples to be described, it is possible to effectively eliminate, particularly, an impurity causing coloration, which often becomes a problem in applications where aspartic acid is polymerized. According to such an embodiment, it is possible to effectively reduce colorants contaminated at a high level with, particularly, a crude product of aspartic acid produced by a fermentation method in which a microbe is used.

The solution (S) is a solution containing aspartic acid or a salt thereof and at least one impurity as described above, and examples of a solvent in which these solutes are dissolved include water, organic solvents such as ethanol and methanol, and mixtures thereof. Additionally, in a case where the method according to the present invention is applied to crude products obtained by a plurality of bioprocesses, such as the fermentation method, or concentrates thereof, since it is normal for a culture medium or culture solution for a microbe to contain water as a solvent, the solution (S) mainly contains water as a solvent component.

In the step (p), regarding the meaning of “the solution (S) containing aspartic acid or a salt thereof and at least one impurity”, this term simply needs to be interpreted literally, and, specifically, a solution enabling crystallization into β-type crystals of aspartic acid to be caused by the adjustment of the pH of the solution (S) to a specific pH value in the acidic region may be employed as the solution (S). Examples of the solution (S) include cultures that are obtained by culturing a variety of cells to be cultured or the like such as microbes capable of generating aspartic acid or a salt thereof (for example, fungi such as bacteria and fungi, blue-green algae, zooplankton, and phytoplankton), an insect cell, an animal cell or a plant cell, treatment products obtained by performing a physical treatment or a chemical treatment (for example, an ultrasonic treatment, a protease treatment or the like) on the above-described culture, reaction products obtained by an enzyme reaction process or a chemical synthesis process that generates aspartic acid or a salt thereof, and biological samples, such as a supernatant, that are obtained by removing a solid component from the above-described culture, treatment product or reaction product by centrifugation or the like.

In recent years, techniques for producing a variety of amino acids using a fermentation method or growth-independent bioprocess in which a genetically modified strain of a bacterium, such as a coryneform bacteria (for example, Corynebacterium glutamicum) or Escherichia coli, is used have been developed. This point makes it possible to conveniently subject a crude product collected from a fermentation method or growth-independent bioprocess in which a bacterium is used to the method according to the present invention. More specifically, a culture that is collected after a bacterium is cultured or reacted in a fermentation method or growth-independent bioprocess, a supernatant obtained by removing a bacterial body from the culture, a treatment product of the culture or supernatant, or a concentrate thereof can be subjected to the step (p) as the solution (S). These biological samples are contaminated with, aside from aspartic acid or a salt thereof, which is considered as a purification subject, a bacterial body, a variety of amino acids derived from a culture medium, a variety of organic acids, proteins, carbohydrates, saccharides, and the like, and, according to the embodiment of the present invention, these impurities can be effectively eliminated. As a result, it is possible to produce high-purity aspartic acid in an α-type crystal form from a biological sample. However, the solution (S) that can be employed in the present invention is not limited to these biological samples.

A case where the concentration of aspartic acid or a salt thereof in the above-described biological sample is not high enough to efficiently generate β-type crystals of aspartic acid through the step (p) is also assumed. In such a case, when a concentration treatment is performed on the biological sample prior to the step (p), a concentrate in which aspartic acid or a salt thereof in a solution sample is concentrated is obtained, and, when the concentrate is employed as the solution (S) in the step (p), β-type crystals of aspartic acid can be efficiently generated through the step (p) within a relatively short time, and thus an embodiment in which such a concentrate is used as the solution (S) in the step (p) can be preferably employed. Additionally, the concentration treatment on the biological sample can be performed by, for example, specifically, vacuum concentration in which a variety of evaporators, vacuum pumps or the like are used, adsorption in which an adsorbent such as activated carbon or silica is used, ultrafiltration, a combination of a variety of these concentration methods or a method in which the above-described method is arbitrarily followed by filtration or the like. However, in the present invention, such a concentration treatment is not essential, and, even in a case where the concentration treatment is employed, the concentration treatment does not limit the above-described configuration.

The concentration of aspartic acid or a salt thereof in the solution (S) that is subjected to the step (p) is not particularly limited as long as β-type crystals of aspartic acid can be generated, and examples thereof include concentration ranges of approximately 0.1 M to approximately 5.0 M, approximately 0.2 M to approximately 4.5 M, approximately 0.5 M to approximately 4.0 M, and approximately 1.0 M to approximately 3.5 M.

Additionally, in a specific embodiment,

a solution sample (preferably, the above-described biological sample or a concentrate thereof) containing
(i) aspartic acid or a salt thereof;
(ii) an amino acid other than aspartic acid and a salt thereof (for example, an amino acid other than aspartic acid or a salt thereof containing at least one selected from the group consisting of glutamic acid, alanine, valine, and a salt thereof); and
(iii) an organic acid or a salt thereof (for example, an organic acid or a salt thereof containing at least one selected from the group consisting of pyruvic acid, malic acid, acetic acid, succinic acid, fumaric acid, and a salt thereof)

can be employed as the solution (S) in the step (p).

Here, it is needless to say that the components (ii) and (iii) correspond to the impurity, and, in the solution (S), the contamination amount of these can be said to be preferably reduced in advance as much as possible. In the embodiment of the present invention, the contamination amount of, especially, the components according to the (ii) and (iii) is intrinsic to a sample to be subjected and is thus not supposed to be actively specified; however, from the viewpoint of the component composition of a biological sample or a concentrated solution thereof, in a specific embodiment, the amount of each component according to the (ii) and (iii) may have the following configuration.

For example, in a specific embodiment, a solution sample containing, in addition to (i) aspartic acid or a salt thereof within a range of a variety of molarities described above, (ii) an amino acid other than aspartic acid or a salt thereof (for example, an amino acid other than aspartic acid or a salt thereof containing at least one selected from the group consisting of glutamic acid, alanine, valine, and a salt thereof) at a molarity within a range of approximately ⅕ to approximately ¾ of the molarity of the aspartic acid or salt there and containing (iii) a variety of organic acids or a salt thereof (for example, an organic acid or a salt thereof containing at least one selected from the group consisting of pyruvic acid, malic acid, acetic acid, succinic acid, fumaric acid, and a salt thereof) at a molarity within a range of approximately ⅕ to approximately ½ of the molarity of the aspartic acid or salt there can be employed as the solution (S) in the step (p).

Furthermore, in another embodiment,

a solution sample (for example, the above-described biological sample or a concentrate thereof) containing
(i) aspartic acid or a salt thereof within a range of a variety of molarities described above;
(ii) at least one selected from the group consisting of glutamic acid, alanine, valine, and a salt thereof (the concentration is, for example, approximately 100 μM to approximately 1 mM, approximately 1 mM to approximately 10 mM, or approximately 10 mM to approximately 1.75 M); and
(iii) at least one selected from the group consisting of pyruvic acid, malic acid, acetic acid, succinic acid, fumaric acid, and a salt thereof (the concentration is, for example, approximately 100 μM to approximately 1 mM, approximately 1 mM to approximately 10 mM, or approximately 10 mM to approximately 1.5 M) may be employed as the solution (S) in the step (p).

In the step (p), in the above-described solution (S), the pH of the solution (S) is adjusted to a predetermined pH value in the acidic region to generate the β-type crystals of aspartic acid. The generation of the β-type crystals of aspartic acid by the adjustment of the pH of this solution (S) is based on the principle of the isoelectric crystallization of aspartic acid, and, specifically, the pH of the solution (S) can be adjusted in the step (p) as described below.

First, an acid or an alkali is added to the solution (S), whereby the pH of the solution (S) is made close to a value of 2.77, which is the isoelectric point of aspartic acid, the solubility of aspartic acid in the solution (S) is decreased, and aspartic acid is crystallized as β-type crystals.

Here, the acid or the alkali is not particularly limited, and, for example, an acid such as hydrochloric acid, sulfuric acid or acetic acid or an alkali such as sodium hydroxide, potassium hydroxide or ammonia water can be used. In a case where the pH of the solution (S) leans to the alkaline side of the isoelectric point (2.77) of aspartic acid, an acid needs to be used to make the pH of the solution (S) close to a value near the isoelectric point. On the other hand, in a case where the pH of the solution (S) leans to the acidic side of the isoelectric point (2.77) of aspartic acid, an alkali needs to be used to make the pH of the solution (S) close to a value near the isoelectric point. In a case where the above-described biological sample or a concentrate thereof is subjected as the solution (S), normally, the pH of the solution (S) leans to be near neutral (pH: 7.0), which is the alkaline side of the isoelectric point of aspartic acid, in almost all cases. Therefore, in such a case, it is normal to use an acid to adjust the pH of the solution (S) in the step (p).

In several embodiments, the pH of the solution (S) that is subjected to the step (p) is approximately 6.00 to approximately 8.00, approximately 6.5 to approximately 7.5, approximately 6.6 to approximately 7.4, approximately 6.7 to approximately 7.3, approximately 6.8 to approximately 7.2, approximately 6.9 to approximately 7.1 and is, for example, approximately 7.0.

The kind of the acid is not particularly limited, but it is convenient to use sulfuric acid as the acid from the viewpoint of easy handling and the cost performance.

In the step (p), the pH of the solution (S) needs to be adjusted to a pH value at which β-type crystals of aspartic acid can be generated, and such a pH value also relies on the concentration of aspartic acid or a salt thereof in the solution (S) and is thus not particularly limited.

For example, in a case where a solution (S) that is a biological sample or concentrate thereof having a pH of near 7.0 (neutral) and has a relatively high concentration (for example, 2.3 M or higher) of aspartic acid or a salt thereof is subjected to the step (p), the present inventors have experimentally confirmed that the generation of β-type crystals of aspartic acid can begin at a point in time where the pH of the solution (S) has been adjusted to be in an acidic region relatively close to 7.0 (neutral) (for example, pH of 4.0 to 6.5) by the addition of an acid, which also relies on a variety of other conditions and thus cannot be said for sure. Therefore, there is no need for the pH of the solution (S) in the step (p) to be adjusted to a value as close to 2.77, which is the isoelectric point of aspartic acid, as possible at all times.

That is, the term “the pH of the solution (S) is adjusted to a predetermined pH value in the acidic region and β-type crystals of the aspartic acid are generated” in the step (p) means that the pH of the solution (S) simply needs to be adjusted to an arbitrary pH value in the acidic region, at which β-type crystals of aspartic acid can be generated, depending on the properties of the solution (S) that is employed in the step (p) or a variety of other conditions. Persons skilled in the art are able to appropriately determine a value to which the pHs of a variety of solutions (S) that are employed in the step (p) are supposed to be adjusted in consideration of the properties of a variety of solutions (S) or a variety of other conditions with reference to what has been disclosed in the present specification.

As described above, the pH value to which the pH of the solution (S) in the step (p) is adjusted is not particularly limited as long as the pH value is a predetermined value in the acidic region at which the generation of β-type crystals of aspartic acid contained in the solution (S) can be attained, and, in a specific embodiment, the pH of the solution (S) can be adjusted to a predetermined value within a range of, for example, approximately 0.50 to approximately 6.95, preferably approximately 1.0 to approximately 6.85 or approximately 1.50 to approximately 4.50, more preferably approximately 2.00 to approximately 4.00, and particularly preferably approximately 2.10 to approximately 3.90.

Additionally, in another embodiment, the pH of the solution (S) in the step (p) can be adjusted to a range of ±2.50, preferably ±2.00, more preferably ±1.50, ±1.00, ±0.90, still more preferably ±0.80, far still more preferably ±0.70, ±0.60, ±0.50, ±0.40, ±0.30, ±0.20, or ±0.10 and particularly preferably ±0.09, ±0.08, ±0.07, ±0.06, ±0.05, ±0.04, ±0.03, ±0.02, or ±0.01 with respect to 2.77, which is the isoelectric point of aspartic acid, and most preferably to 2.77, which is the isoelectric point of aspartic acid. According to such an embodiment, since the step (p) is performed by adjusting the pH of the solution (S) with a standard of 2.77, which is the isoelectric point of aspartic acid, it becomes possible to effectively reduce or eliminate a variety of impurities other than aspartic acid from the solution (S) while holding a high collection rate of β-type crystals of aspartic acid thanks to a difference in isoelectric point between aspartic acid and a variety of impurities described above.

The amount of the acid or alkali added to the solution (S) needs to be adjusted as appropriate in consideration of a variety of conditions including the initial pH value or target pH value of the solution (S) and is not particularly limited. In several embodiments, the amount of the acid or alkali added to the solution (S) may be set within a range of, for example, approximately 50 parts by mass to approximately 200 parts by mass or approximately 60 parts by mass to approximately 150 parts by mass with respect to approximately 100 parts by mass of aspartic acid in the solution (S).

Furthermore, in a specific embodiment, the step (p) can be performed in the presence of a predetermined seed crystal to accelerate the growth of β-type crystals of aspartic acid, and, in a case where the step (p) is performed in the presence of the seed crystal, a predetermined amount of the seed crystal needs to be added to the solution (S) prior to the adjustment of the pH of the solution (S). The seed crystal is not limited as long as the seed crystal accelerates the generation of β-type crystals of aspartic acid, but it is preferable to contain β-type crystals of aspartic acid to reliably generate the β-type crystals. In this case, regarding the β-type crystals of aspartic acid as the seed crystal, the seed crystal does not need to be purified to a high purity at all times and may be a crude crystal sample containing impurities other than β-type crystals of aspartic acid. For example, a crude crystal sample of aspartic acid generated by the isoelectric crystallization (the step (p)) performed using a biological sample or a concentrate thereof as a starting material without adding the seed crystal may also be used as the seed crystal.

The amount of the seed crystal in the solution (S) needs to be adjusted as appropriate depending on other crystallization conditions and is not particularly limited. The seed crystal can be added to the solution (S) within a range of, for example, approximately 0.001 parts by mass to approximately 5.00 parts by mass and preferably approximately 0.001 parts by mass to approximately 4.00 parts by mass in a specific embodiment, or, for example, approximately 0.01 parts by mass to approximately 3.00 parts by mass, preferably approximately 0.01 parts by mass to approximately 2.50 parts by mass and particularly preferably approximately 0.01 parts by mass to approximately 2.00 parts by mass in a specific embodiment in another embodiment with respect to approximately 100 parts by mass of aspartic acid or a salt thereof in the solution (S). However, in the present invention, originally, the addition of the seed crystal to the solution (S) is not essential at all times. As also shown in the examples to be described below, without the addition of the seed crystal, it is possible to generate desired β-type crystals of aspartic acid in the step (q) even, and it is possible to produce desired α-type crystals of aspartic acid in the end through the subsequent step (r).

In the step (p), there are also cases where heat of reaction such as heat of dilution or heat of dissolution is generated at the time of adding the acid or the alkali to the solution (S), and it is convenient to add the acid or the alkali stepwise to avoid the generation of the heat and realize a uniform crystallization reaction. Additionally, while not particularly limited, in a case where the temperature of the solution (S) has been increased due to the generation of the heat of reaction immediately after the adjustment of the pH of the solution (S) in the step (p), heat may be dissipated until normal temperature, and the solution (S) may be further cooled to a temperature range of 2° C. to 10° C. (for example, approximately 4° C.). However, this heat dissipation step or cooling step is not an element affecting the generation of aspartic acid or a salt thereof in a crystal form predetermined by the present invention or the elimination of impurities and is not an essential configuration element of the present invention.

Furthermore, it is also possible to employ an embodiment in which the temperature of the solution (S) is controlled to a predetermined temperature range at the time of adding the acid or the alkali to the solution (S) in the step (p), which is not essential at all times. In such an embodiment, the temperature of the solution (S) can be controlled to be, for example, within a range of approximately 30° C. to approximately 190° C., preferably approximately 35° C. to approximately 150° C., more preferably approximately 40° C. to approximately 110° C., still more preferably approximately 45° C. to approximately 105° C., and far still more preferably approximately 50° C. to approximately 105° C. Furthermore, in the case of controlling the temperature of the solution (S) under a normal pressure condition, the temperature of the solution (S) can be controlled to be, for example, within a range of approximately 45° C. to approximately 100° C., preferably approximately 50° C. to approximately 100° C., more preferably approximately 60° C. to approximately 100° C., still more preferably approximately 65° C. to approximately 100° C., far still more preferably approximately 70° C. to approximately 100° C., particularly preferably approximately 75° C. to approximately 100° C., and most preferably approximately 78° C. to approximately 100° C. According to an embodiment in which the temperature of the solution (S) is controlled to be within such a predetermined temperature range, it becomes possible to produce aspartic acid having a uniform quality with favorable reproducibility, and, since the temperature range is close particularly to 100° C., it is possible to expect an effect of reducing amino acids other than aspartic acid at a higher level, and thus an embodiment in which the temperature of the solution (S) is controlled to be within a variety of temperature ranges described above can be preferably employed. Furthermore, an embodiment in which the temperature of the solution (S) is controlled to be within a relatively low temperature range, for example, approximately 30° C. to approximately 100° C., preferably approximately 30° C. to approximately 80° C. and more preferably approximately 40° C. to approximately 70° C. or the like at the time of controlling the temperature of the solution (S) to be within a predetermined temperature range may also be employed. According to such an embodiment, for example, in a case where the proportion of a contamination component other than aspartic acid or a salt thereof that is contained in the solution (S) is relatively small or the like, it is possible to realize a more efficient process by reducing energy that is injected into steps to the minimum necessary level, and thus the embodiment can be preferably employed.

As described above, when the pH of the solution (S) is adjusted to a predetermined pH value within the acidic region to generate β-type crystals of aspartic acid in the solution (S), the majority of impurities other than aspartic acid remain in a liquid fraction (that is, the supernatant with respect to a crude crystal solid fraction) of the solution (S). Therefore, the majority of impurities that contaminate the solution (S) can be eliminated by separating a fraction containing the generated β-type crystals from the solution (S) after the β-type crystals of aspartic acid are generated in the solution (S) in the step (p).

In the present invention, as a method for separating a fraction containing the generated β-type crystals from the solution (S), any method can be employed with no particular limitations as long as at least a part of the impurities that remain in the liquid portion of the solution (S) is eliminated. In several embodiments, it is possible to employ, for example, i) a method in which a fraction containing at least a part of β-type crystals of aspartic acid generated in the solution (S) is separated by a method such as suction or ii) a method in which a crude crystalline fraction (solid fraction) generated in the solution (S) is precipitated or the like, then, a supernatant liquid portion is eliminated by a method such as suction, and a fraction containing at least a part of the remaining β-type crystals of aspartic acid is extracted.

In this case, as long as at least a part of the impurities that remain in the liquid fraction of the solution (S) is eliminated, since a purpose of purifying aspartic acid is understood to be attained to a certain extent, the fact that a part of impurities is brought into a fraction containing at least a part of β-type crystals of aspartic acid that are obtained in the step (p) is not excluded. Even when a considerable amount of impurities is brought into the fraction containing at least a part of β-type crystals of aspartic acid in the step (p), it is possible to expect that the impurities are further reduced or eliminated through the subsequent step (q).

Furthermore, in a specific embodiment, a method in which a crude crystalline fraction containing β-type crystals of aspartic acid is separated from the solution (S) from which the β-type crystals of aspartic acid have been generated using a variety of solid-liquid separation methods such as evaporation, filtration, suction filtration, and vacuum drying may also be employed. According to an embodiment in which such a solid-liquid separation method is employed, since it is possible to almost completely eliminate the supernatant portion where the impurities remain and effectively eliminate the impurities, impurities that are brought into the crude crystalline fraction containing β-type crystals of aspartic acid to be obtained can be significantly reduced, and thus the embodiment according to the present invention can be preferably employed.

On the “fraction containing at least a part of β-type crystals of aspartic acid” or “crude crystalline fraction containing β-type crystals of aspartic acid” separated from the solution (S), a washing step using a solvent such as water and a subsequent drying step may be performed arbitrarily or an arbitrary combination of these washing step and drying step may be repeated a plurality of times.

Hitherto, the step (p) that can be employed prior to the step (q) in a specific embodiment of the present invention has been described in detail, and, in the case of employing an embodiment in which the step (p) is performed prior to the step (q), in the step (q) to be described in detail below, it is possible to prepare “a slurry containing a crystalline fraction (X) containing β-type crystals of aspartic acid” using the fraction or crude crystalline fraction containing β-type crystals of aspartic acid generated in the step (p).

### Step (q)

Next, the step (q) will be described.

The step (q) is “to prepare a slurry containing a crystalline fraction (X) containing β-type crystals of aspartic acid and at least one impurity” as described above.

Here, “the crystalline fraction (X) containing β-type crystals of aspartic acid and at least one impurity” simply needs to be interpreted literally, and the meanings of the terms “β-type crystals of aspartic acid” and “impurity” are as described above. However, “the crystalline fraction (X) containing β-type crystals of aspartic acid and at least one impurity” is not always limited to the “fraction containing at least a part of β-type crystals of aspartic acid” or “crude crystalline fraction containing β-type crystals of aspartic acid” that is obtained in the step (p) or the fraction on which a predetermined treatment has been performed, and crystal samples obtained by other procedures can also be subjected to the step (q).

Additionally, regarding “the crystalline fraction (X) containing β-type crystals of aspartic acid and at least one impurity” in the step (q), in a case where a predetermined sample obtained by the step (p) or other procedures already has a slurry form and can be subjected to the subsequent step (r), a form in which the sample is prepared as “the slurry of the crystalline fraction (X)” as it with no treatments performed thereon and subjected to the step (r) as it is also included in “the preparation of the slurry of the crystalline fraction (X)” in the step (q). On the other hand, in a case where a predetermined sample obtained by the step (p) or other procedures has a form of a suspension or slurry of crude crystals at the time of the obtainment, “the preparation of the slurry of the crystalline fraction (X)” may be prepared by separating a supernatant and a crude crystalline fraction by a variety of solid-liquid separation methods such as evaporation, filtration, suction filtration, and vacuum drying, arbitrarily performing a washing treatment using a solvent such as water and a drying treatment on the separated crude crystalline fraction, and re-suspending the obtained crude crystalline fraction in a solvent such as water. Furthermore, in a case where a previously obtained sample has, for example, a form of a solid matter or semi-solid matter of crude crystals rather than a slurry form, it is needless to say that the preparation of “the slurry of the crystalline fraction (X)” by suspending the sample in an arbitrary solvent such as water is also included in the step (q). Additionally, even when a previously obtained sample already has a slurry form, the preparation of “the slurry of the crystalline fraction (X)” by re-suspending crude crystals obtained by separating a solid fraction (crude crystalline fraction) by a variety of solid-liquid separation methods and then arbitrarily performing a washing treatment or a drying treatment on the separated solid fraction (crude crystalline fraction) in a solvent such as water is also included in the step (q). Alternatively, “the slurry of the crystalline fraction (X)” may be prepared by further diluting a sample of a previously obtained crude crystal slurry with a solvent such as water, and such a procedure is also included in the step (q).

That is, “the slurry of the crystalline fraction (X)” in the step (q) simply needs to be interpreted literally and needs to be understood as a term meaning a mixture in which a crystal component is excessively present in a solvent beyond the saturation solubility and crystal particles are suspended in the solvent rather than a crystal solution in which a crystal component is completely dissolved. The saturation solubility of the crystal component also relies on temperature or the like and thus cannot be said for sure, but the concentration of the crystalline fraction (X) in the slurry can be set to, for example, approximately 10 to 70 w/v%, preferably approximately 15 to 60 w/v%, and more preferably 20 to 50 w/v%. However, the saturation solubility is not limited to these ranges. The kind of the solvent is not particularly limited, but water (for example, ion exchange water, pure water, or ultrapure water) is preferable from the viewpoint of easy handling.

In an embodiment in which the slurry of the crystalline fraction (X) is prepared using a solid matter of crude crystals containing β-type crystals of aspartic acid in the step (q), the slurry of the crystalline fraction (X) may be prepared by suspending the solid matter of crude crystals in a certain amount of a predetermined solvent such as water and preparing crude crystal slurries within a variety of concentration ranges described above. In this case, the crystalline fraction (X) is preferably prepared by suspending the solid matter of crude crystals in a solvent substantially composed of water, and the meaning of “a solvent substantially composed of water” means that inevitable contamination by a solvent substance other than water is not excluded.

“The slurry of the crystalline fraction (X)” in the step (q) may contain, aside from β-type crystals of aspartic acid, impurities that are supposed to be eliminated or reduced by the subsequent step (r). The impurities that “the slurry of the crystalline fraction (X)” may contain can be said to be a substance that intrinsically contaminates a crude crystal sample, which is considered as a purification subject in the step (r), and thus the kind or contamination amount thereof is not limited.

Additionally, as described regarding the solution (S), the amount of the impurities that contaminate the crystalline fraction (X) or the slurry thereof is preferably as small as possible; however, in a case where the crystalline fraction (X) or the slurry thereof is derived from a biological sample, a concentrate thereof or the like, contamination with a variety of amino acids other than aspartic acid that are derived from a bacterial body or a culture medium or salts thereof, a variety of organic acids or salts thereof, a protein, a carbohydrate, a saccharide, or the like is assumed.

Regarding this point, in a specific embodiment, “the slurry of the crystalline fraction (X)” in the step (q) is derived from a biological sample, a concentrate thereof or the like and, more specifically, may contain the following component composition.
(i) Aspartic acid at a concentration at which the crude crystal slurry can be formed (for example, approximately 0.05 M to approximately 4.5 M, approximately 0.8 M to approximately 4.0 M, or approximately 1.0 M to approximately 3.5 M);
(ii) an amino acid other than aspartic acid or a salt thereof containing at least one selected from the group consisting of glutamic acid, alanine, valine, and a salt thereof (the concentration is, for example, approximately 0.05 mM to approximately 1.0 M, approximately 0.1 mM to approximately 1.0 M, approximately 1 mM to approximately 1.0 M, approximately 1 mM to approximately 800 mM, approximately 1 mM to approximately 500 mM, or approximately 1 mM to approximately 100 mM); and
(iii) an organic acid or a salt thereof containing at least one selected from the group consisting of pyruvic acid, malic acid, acetic acid, succinic acid, fumaric acid, and a salt thereof (the concentration is, for example, approximately 0.05 mM to approximately 1.0 M, approximately 0.1 mM to approximately 1.0 M, approximately 1 mM to approximately 1.0 M, approximately 1 mM to approximately 800 mM, approximately 1 mM to approximately 500 mM, or approximately 1 mM to approximately 100 mM).

Several embodiments of the step (q) have been exemplified as described above. In the step (q), it is simply required to prepare “the slurry of the crystalline fraction (X)” that can be subjected to the subsequent step (r), and specific forms thereof are not limited within the scope of the literal meaning of this term.

### Step (r)

The step (r) is a step of heating “the slurry of the crystalline fraction (X)” prepared in the step (q) to convert the β-type crystals of aspartic acid that are contained in the slurry to α-type crystals and then obtain a crystalline fraction (Y) containing aspartic acid in the α-type crystals.

The term “heating the slurry to convert the β-type crystals of aspartic acid to a-type crystals” in the step (r) refers to a crystal conversion from the β-type crystals of aspartic acid in the slurry to α-type crystals by a heating treatment on the slurry of the crystalline fraction (X) and may conceptually include not only an aspect in which the crystal conversion is caused in the middle of the heating treatment on the slurry but also an aspect in which the crystal conversion is caused after the heating treatment (for example, in the middle of or after a treatment of leaving the sample to stand or cooling the sample).

In the step (r), conditions such as the heating temperature, the heating time, the pressurization condition and the like at the time of heating the slurry of the crystalline fraction (X) are not particularly limited as long as a desired crystal conversion is caused. The heating temperature can be set to a range of, for example, approximately 60° C. to approximately 190° C., approximately 61° C. to approximately 190° C., approximately 62° C. to approximately 190° C., approximately 63° C. to approximately 190° C., approximately 64° C. to approximately 190° C., approximately 65° C. to approximately 190° C., preferably approximately 65° C. to approximately 150° C., more preferably approximately 65° C. to approximately 110° C., approximately 66° C. to approximately 110° C., approximately 67° C. to approximately 110° C., more preferably approximately 68° C. to approximately 110° C., approximately 69° C. to approximately 100° C. Additionally, in the case of heating the slurry of the crystalline fraction under a normal pressure condition, the heating temperature can be set to a range of, for example, approximately 60° C. to approximately 100° C., approximately 61° C. to approximately 100° C., approximately 62° C. to approximately 100° C., approximately 63° C. to approximately 100° C., approximately 64° C. to approximately 100° C., more preferably approximately 65° C. to approximately 100° C., approximately 66° C. to approximately 100° C., approximately 67° C. to approximately 100° C., still more preferably approximately 68° C. to approximately 100° C., approximately 69° C. to approximately 100° C.

In still another embodiment, a heating temperature in a relatively low temperature range may be employed, for example, a temperature range of approximately 30° C. to approximately 190° C., approximately 35° C. to approximately 190° C., approximately 36° C. to approximately 190° C., approximately 37° C. to approximately 190° C., approximately 38° C. to approximately 190° C., approximately 39° C. to approximately 190° C., approximately 40° C. to approximately 190° C., preferably approximately 30° C. to approximately 150° C., approximately 35° C. to approximately 150° C., approximately 36° C. to approximately 150° C., approximately 37° C. to approximately 150° C., approximately 38° C. to approximately 150° C., approximately 39° C. to approximately 150° C., approximately 40° C. to approximately 150° C., approximately 60° C. to approximately 150° C., more preferably approximately 30° C. to approximately 110° C., approximately 35° C. to approximately 110° C., approximately 36° C. to approximately 110° C., approximately 37° C. to approximately 110° C., approximately 38° C. to approximately 110° C., approximately 39° C. to approximately 110° C., approximately 40° C. to approximately 110° C., or approximately 60° C. to approximately 110° C. may also be employed, and, furthermore, in the case of heating under a normal pressure condition, a temperature range of approximately 30° C. to approximately 100° C., approximately 35° C. to approximately 100° C., approximately 36° C. to approximately 100° C., approximately 37° C. to approximately 100° C., approximately 38° C. to approximately 100° C., approximately 39° C. to approximately 100° C., or approximately 40° C. to approximately 100° C. may also be employed.

Additionally, in several embodiments, the heating temperature can also be set to a range of, for example, approximately 70° C. to approximately 100° C., approximately 75° C. to approximately 100° C., approximately 78° C. to approximately 100° C., approximately 80° C. to approximately 100° C., approximately 85° C. to approximately 100° C., approximately 88° C. to approximately 100° C., approximately 90° C. to approximately 100° C., or approximately 95° C. to approximately 100° C. In the case of heating under a normal pressure condition, as the temperature range becomes closer to 100° C., impurities such as amino acids other than aspartic acid, such as glutamine or alanine can be reduced or eliminated at a higher level, and thus embodiments based on such temperature ranges can be preferably employed.

Furthermore, the heating time may be set as appropriate to a range in which a desired crystal conversion is caused depending on the properties of the slurry of the crystalline fraction (X), the heating condition or the like and is not particularly limited. Ordinarily, in the case of heating under a normal pressure condition, as the temperature range becomes closer to 100° C., α-type crystals are generated from the β-type crystals within a relatively short time, and, on the other hand, in a case where a relatively low heating temperature is employed, there is a tendency that a relatively long heating time is required until α-type crystals are generated from the β-type crystals. For example, the lower limit value of the heating time can be set to, for example, five minutes or more, preferably approximately 10 minutes or more, approximately 15 minutes or more or approximately 30 minutes or more from when the temperature of the sample reaches a predetermined heating temperature. In another embodiment, the heating time can also be set to, for example, approximately one hour or more, approximately two hours or more or approximately three hours or more from when the temperature of the sample reaches a predetermined heating temperature so that α-type crystals of aspartic acid are reliably obtained. The upper limit value of the heating time may be set depending on a variety of conditions so that a predetermined amount of α-type crystals of aspartic acid are generated, is not particularly limited, and can be set to, for example, approximately 20 hours, approximately 15 hours, approximately 10 hours, or approximately five hours.

Each numerical range obtained by arbitrarily combining each lower limit value and each upper limit value of the above-described heating times is a range of the heating time that can be employed in a specific embodiment and is clearly expressed as an embodiment in the present specification. Additionally, the heating of the slurry of the crystalline fraction (X) in the step (r) may also be performed under a pressurization condition as long as α-type crystals can be generated from the β-type crystals.

After the heating treatment on the slurry of the crystalline fraction (X) is completed in the step (r) as described above, the sample may be left to stand until the sample reaches normal temperature and may be further cooled to a temperature range of 2° C. to 10° C. (for example, approximately 4° C.), which does not particularly limit the configuration. It is needless to say that not only an aspect in which the crystal conversion is caused in the middle of the heating treatment as described above but also an aspect in which the crystal conversion is caused while the crystal sample is left to stand or cooled as described above after the heating treatment can be included in the present invention.

As described above, in the step (r), “the slurry of the crystalline fraction (X)” prepared in the step (q) is heated, and the β-type crystals of aspartic acid that are contained in the slurry are converted to α-type crystals.

In the slurry of the crystalline fraction (X), α-type crystals of aspartic acid are generated by the above-described heating treatment, and then a crude crystalline fraction (Y) containing the α-type crystals is obtained.

Here, the meaning of “a crude crystalline fraction (Y) containing the α-type crystals of aspartic acid is obtained” is as described below.

When the β-type crystals of aspartic acid are converted to α-type crystals by the heating treatment in the slurry of the crystalline fraction (X) as described above, at least a part of impurities contaminating the slurry is put into a state of being dissolved in a liquid fraction (that is, a supernatant with respect to the crude crystal solid fraction).

Then, a crystalline fraction containing at least the generated α-type crystals is separated from the entire crystalline slurry in which the α-type crystals of aspartic acid have been generated by the heating treatment in the step (r), whereby the majority of the impurities contaminating the crystalline fraction (X) or the slurry thereof can be eliminated.

A method for separating the crystalline fraction containing the generated α-type crystals from the entire slurry on which the heating treatment has been performed needs to be an aspect in which at least a part of impurities that remain in a liquid portion of the slurry is eliminated and is not particularly limited. In several embodiments, for example, a method in which a fraction containing at least a part of the generated α-type crystals of aspartic acid in the slurry is separated by a method such as suction or a method in which a crystalline fraction (solid fraction) is precipitated, centrifuged or the like, then, a supernatant liquid portion is eliminated by a method such as suction, and a fraction containing at least a part of the remaining α-type crystals of aspartic acid is extracted can be employed. In this case, as long as at least a part of the impurities that remain in the supernatant liquid fraction is eliminated, since the purification of aspartic acid can be said to be attained to a certain extent, the fact that a part of the impurities is brought into a fraction containing at least a part of α-type crystals of aspartic acid that are obtained is not excluded.

Furthermore, in another embodiment, a method in which the crystalline fraction containing the α-type crystals of aspartic acid is separated from the crystalline slurry in which the α-type crystals of aspartic acid have been generated by a variety of solid-liquid separation methods such as evaporation, filtration, suction filtration, and vacuum drying can be employed. According to an embodiment in which such a solid-liquid separation method is employed, since it is possible to almost completely eliminate a supernatant liquid portion where the impurities remain and effectively eliminate the impurities, impurities that are brought into the crystalline fraction containing the α-type crystals of aspartic acid to be obtained can be significantly reduced. Therefore, such an embodiment can be preferably employed in the present invention.

As described above, in the step (r), the intended α-type crystals of aspartic acid are produced.

### Other Steps, Conditions or the Like

At least a part or all of the steps (p), (q), and (r) may be executed using an appropriate tool or device depending on the amount of α-type crystals of aspartic acid intended to be produced. For example, for the isoelectric crystallization in the step (p) and the heating treatment in the step (r), it is simply required to select and use an arbitrary heating device as appropriate. Specifically, a heating device may be selected as appropriate depending on an intended production scale. For example, in a case where laboratory-scale production is intended, a commercially available beaker or hot stirrer, which are also employed in the following examples, can also be used, which does not intend to particularly limit the configuration. On the other hand, in a case where industrial scale production is intended, it is possible to use a general-purpose or exclusive reactor or to design a reaction tank or a heating device configuring a plant and execute at least a part or all of the steps (p), (q), and (r) using this tank or device. In other words, the method according to the present invention also includes embodiments that are realized by a variety of configurations such as a combination of a variety of laboratory-scale devices, a combination of a variety of reactors or heating devices, or a large-scale production plant.

Furthermore, while not always essential, the method according to the present invention may arbitrarily include a step of confirming whether or not β-type columnar crystals have been generated by visual or microscopic observation and/or an X-ray diffraction method regarding intermediate products such as the solution (S) after the step (p) and the crystalline fraction (X) obtained by the step (p). Furthermore, the method according to the present invention may arbitrarily include a step of confirming whether or not α-type plate-like crystals have been generated by the above-described method regarding the crude crystal slurry after the heating treatment in the step (r) and/or the crystalline fraction (Y) obtained by the step (r).

Additionally, while not always essential, the method according to the present invention may include, in all or part of the steps (p) to (r), a step of monitoring the residual amount, residual rate, elimination rate or the like of the impurities in the sample at an arbitrary point in time by a variety of chemical analysis methods such as HPLC as shown in the following examples.

Hitherto, specific embodiments of the present invention have been described in detail, but it is needless to say that the present invention is not limited to the above-described embodiments. A variety of modifications, corrections, and combinations can be employed regarding each configuration, element and characteristic within the scope of the gist of the present invention.

In the present invention, the terms “include”, “contain”, and “have” do not exclude the presence of an element other than elements that are mentioned as the objects of these terms, and these terms are used interchangeably.

In addition, in the present specification, “to” means equal to or more than a value shown before the expression “to” and equal to or less than a value shown after the expression “to.” Additionally, in a case where the term “approximately” is used to express numerical ranges and numerical values shown in the present specification, it is needless to say that numerical ranges and numerical values excluding the term are also considered to be clearly expressed in the present specification as elements capable of configuring the embodiments according to the present invention.

Hereinafter, the present invention will be more specifically described by showing examples and comparative examples, but the present invention is not limited to the examples.

### EXAMPLES

### Test Example 1

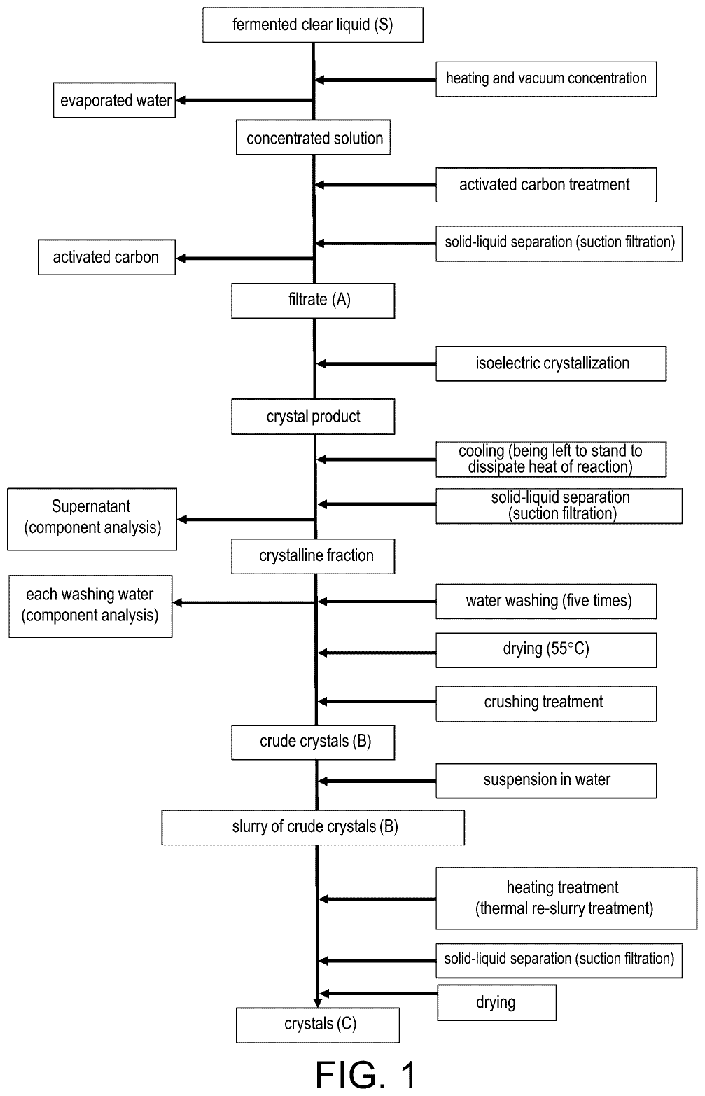

The present test example is an example in which a genetically modified Corynebacterium glutamicum capable of producing aspartic acid (hereinafter, referred to as an Asp-producing Corynebacterium in some cases) was cultured in a predetermined reaction culture medium and α-type crystals of aspartic acid were produced using a fermented clear liquid of a culture obtained by the above-described culturing as a starting material according to, schematically, a procedure shown in FIG. 1. Hereinafter, each procedure will be described in detail.

### (1) Concentration/Activated Carbon Treatment

A modified Corynebacterium glutamicum strain modified by introducing an enzyme gene capable of participating in an L-aspartic acid metabolic pathway so as to be capable of efficiently producing L-aspartic acid was cultured in a predetermined reaction culture medium to generate aspartic acid in the reaction culture medium. 5 L of a fermented clear liquid (S) obtained by eliminating a bacterial body from the obtained culture was subjected to the following step procedure.

Vacuum concentration was performed on 5 L of the fermented clear liquid (S) using a flash evaporator (manufactured by Tokyo Rikakikai Co., Ltd., model No. MF-10B), a diaphragm vacuum pump (manufactured by Tokyo Rikakikai Co., Ltd., model No. EVP-1200) and a vacuum controller (Tokyo Rikakikai Co., Ltd., model No. NVC-2200). Next, 4 g of powdered activated carbon (“CARBORAFFIN” manufactured by Osaka Gas Chemicals Co., Ltd.) per 100 g of aspartic acid was added to the obtained concentrated solution and stirred at normal temperature for 70 minutes, a solution was separated into activated carbon and 1400 mL of a filtrate (A) by a suction filtration method, and 1400 mL of the filtrate (A) obtained as described above was subjected to isoelectric crystallization to be described below.

From the fact that 5 L of the fermented clear liquid (S) was concentrated into 1400 mL of the filtrate (A), the final concentration of aspartic acid in this filtrate (A) is estimated to be 2.5 M according to the calculation.

### (2) Isoelectric Crystallization

0.3 g of crude crystals obtained by a method to be described below was added to the filtrate (A) in advance as seed crystals, and 300 g of sulfuric acid was slowly added to an obtained solution at normal temperature under stirring, thereby adjusting the pH of the solution to near 2.77, which corresponds the isoelectric point of aspartic acid. For the measurement of the pH of the solution, a pH meter (manufactured by Horiba, Ltd., Model No. D-71) was used. As a result of an isoelectric crystallization treatment by the pH adjustment of the solution, a crystalline fraction was generated in the solution. At the time of the pH adjustment of the solution, since the temperature increased up to approximately 70° C. due to the generation of heat by a neutralization reaction, the solution was left to stand under stirring until normal temperature was reached, then, the stirring was stopped, and the solution was cooled at 4° C.

The crude crystals added to the filtrate (A) as the seed crystals were obtained in advance as described below. That is, the same isoelectric crystallization was performed on a concentrated solution (filtrate) derived from a fermented clear liquid obtained in the same manner as described above except that no seed crystals were added thereto, crude crystals were generated, a sample in which as large columnar crystals as possible had been generated was selected in advance, and the sample was used as the seed crystals.

(3) Crystal separation

Solid-liquid separation was performed on a crystal product obtained by the above-described isoelectric crystallization by the suction filtration method, thereby obtaining a solid crystalline fraction. This solid crystalline fraction was washed by pouring 1750 mL of ultrapure water from above to eliminate impurities attached to crystal surfaces. Furthermore, the same washing step was repeated four times, and wet crude crystals were obtained. The obtained wet crude crystals were moved to a stainless steel square tray, injected into a constant temperature dryer (manufactured by AS ONE Corporation, model No. OFW-300B) and dried at 55° C. Furthermore, the dried crystal sample was crushed using a mixer (manufactured by Hanwa Co., Ltd., model No. BKE-07), and the crystal sample was collected in a plastic container. The weight of the obtained crude crystal sample (B) was 460 g.

### (4) Heating Treatment (Thermal Re-Slurry Treatment)

90.0 g of the sample of crude crystals (B) obtained in the section (3) was weighed with an electronic balance (manufactured by Shimadzu Corporation, model No. UW6200H) and suspended in ultrapure water so that the final volume reached 300 mL, thereby preparing a 30% crude crystal slurry. This crude crystal slurry was stirred and heated in a beaker using a hot stirrer (manufactured by AS ONE Corporation, model No. HS-360H). The heating was stopped after 10 minutes from when the temperature of the sample reached 100° C., and the sample was cooled to normal temperature under stirring. After that, the stirring was stopped, and the sample was cooled at 4° C.

### (5) Crystal Separation

A solid-liquid separation treatment was performed on a thermal re-slurry liquid obtained as described above by a suction filtration method, and the thermal re-slurry liquid was separated into a supernatant and a solid crystalline fraction. 100 mL of ultrapure water was poured over the obtained solid crystalline fraction to wash crystals and eliminate impurities attached to the surfaces. Wet crude crystals after the washing were moved to a stainless steel square tray, injected into the constant temperature dryer (manufactured by AS ONE Corporation, model No. OFW-300B) and dried at 55° C. Furthermore, the dried crystal sample was crushed using the mixer (manufactured by Hanwa Co., Ltd., model No. BKE-07), and the crystal sample was collected in a plastic container. The weight of the obtained crystals (C) was 71.4 g.

### (6) Various Analyses

In each of the steps described above, a part of each of the fermented clear liquid (S), the concentrated solution, the filtrate (A), the crude crystals (B), and the crystals (C) was extracted and subjected to a variety of amino acid analyses and a variety of organic acid analyses. A part of each of the crude crystals (B) and the crystals (C) was dissolved in a sodium hydroxide (manufactured by FUJIFILM Wako Pure Chemical Corporation) aqueous solution at a concentration of 100 g/L to produce an analysis sample.

Specifically, in a variety of the amino acid analyses, the fermented clear liquid (S) was diluted 1000 times, the concentrated solution and the filtrate (A) were diluted 2500 to 4000 times, and the crude crystals (B) and the crystals (C) were diluted 1000 times using a sodium citrate buffer having a pH of 2.2 (manufactured by FUJIFILM Wako Pure Chemical Corporation), and each diluted sample was analyzed using a high performance liquid chromatography system (Shimadzu Corporation, Prominence). On the other hand, in a variety of the organic acid analyses, the fermented clear liquid (S) was diluted 100 times, the concentrated solution and the filtrate (A) were diluted 250 to 400 times, and the crude crystals (B) and the crystals (C) were diluted 20 times using 0.75 mM sulfuric acid (manufactured by FUJIFILM Wako Pure Chemical Corporation), and each diluted sample was analyzed using the high performance liquid chromatography system (manufactured by Shimadzu Corporation, Prominence).

Additionally, a part of the sample was extracted in the middle of the present heating treatment (thermal re-slurry method) and at a point in time of each of the following (b) to (d), and each of the extracted samples was observed with a microscope (manufactured by Olympus Corporation, model No. CX41LF).
(b) A point in time where the sample temperature reaches 70° C. after the heating begins (before a crystal conversion)
(c) A point in time where the sample temperature reaches 77° C. after the heating begins (in the middle of a crystal conversion)
(d) A point in time where the sample temperature reaches 100° C. after the heating begins (after a crystal conversion)

Furthermore, regarding each sample of the crude crystals (B) and the crystals (C), the crystal structure of each sample was analyzed by X-ray diffraction (manufactured by Rigaku Corporation, X-ray diffractometer SmarLab) according to a normal method.

### Results

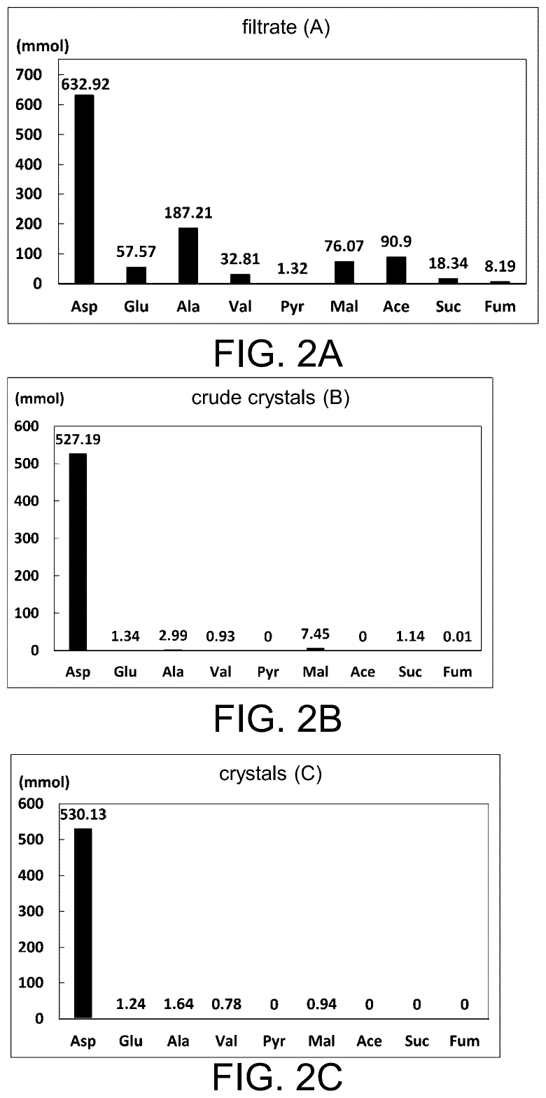

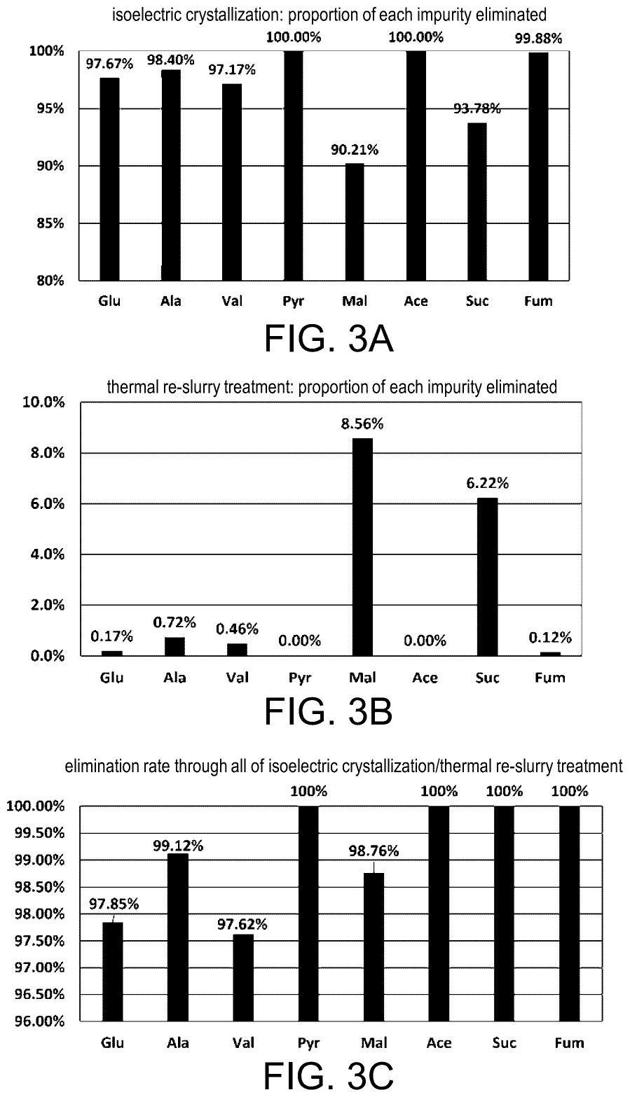

The results of the amino acid analyses and the organic acid analyses are shown in Tables 1 and 2 and FIGS. 2A to 2C and FIG. 3A to 3C.

TABLE 1

| | | | Concentrated solution | | Filtrate (A) | | | | | |
|---|---|---|---|---|---|---|---|---|---|---|
| | | | (before activated | | (sample to be | | | | | |
| | | | carbon | | subjected to | | | | | |
| | Fermented clear | | treatment/suction | | isoelectric | | | | Crystals (C) | |
| | liquid (S) | | filtration) | | crystallization) | | Crude crystals (B) | | (final product) | |
| | | Total | | Total | | Total | | Total | | Total |
| | Concentration | amount | Concentration | amount | Concentration | amount | Concentration | amount | Concentration | amount |
| | (mmol/L) | (mmol) | (mmol/L) | (mmol) | (mmol/L) | (mmol) | (mmol/g) | (mmol) | (mmol/g) | (mmol) |
| Aspartic | 669.00 | 655.40 | 2312.57 | 634.35 | 2366.51 | 632.92 | 5.86 | 527.19 | 7.43 | 530.13 |
| acid (Asp) | | | | | | | | | | |
| Glutamic | 59.28 | 58.07 | 202.70 | 55.60 | 215.27 | 57.57 | 0.01 | 1.34 | 0.02 | 1.24 |
| acid (Glu) | | | | | | | | | | |
| Alanine | 195.33 | 191.36 | 667.10 | 182.99 | 699.99 | 187.21 | 0.03 | 2.99 | 0.02 | 1.64 |
| (Ala) | | | | | | | | | | |
| Valine | 34.74 | 34.04 | 117.54 | 32.24 | 122.67 | 32.81 | 0.01 | 0.93 | 0.01 | 0.78 |
| (Val) | | | | | | | | | | |
| Pyruvic | 1.73 | 1.70 | 1.86 | 0.51 | 4.94 | 1.32 | 0.00 | 0.00 | 0.00 | 0.00 |
| acid (Pyr) | | | | | | | | | | |
| Malic | 80.62 | 78.98 | 283.09 | 77.65 | 284.43 | 76.07 | 0.08 | 7.45 | 0.01 | 0.94 |
| acid (Mal) | | | | | | | | | | |
| Acetic | 105.69 | 103.54 | 349.47 | 95.86 | 339.88 | 90.9 | 0.00 | 0.00 | 0.00 | 0.00 |
| acid (Ace) | | | | | | | | | | |
| Succinic | 23.74 | 23.26 | 77.86 | 21.36 | 68.56 | 18.34 | 0.01 | 1.14 | 0.00 | 0.00 |
| acid (Suc) | | | | | | | | | | |
| Fumaric | 7.62 | 7.47 | 31.22 | 8.56 | 30.61 | 8.19 | 0.00 | 0.01 | 0.00 | 0.00 |
| acid (Fum) | | | | | | | | | | |

*The crude crystals (B) and the crystals (C) were measured by adjusting an analysis sample to 1 g/10 mL and subjecting the sample to HPLC analysis.

TABLE 2

| | | | Elimination rate by | |
|---|---|---|---|---|
| | | Elimination rate by | both of isoelectric | |
| | Elimination rate by | thermal re-slurry | crystallization/thermal | |
| | isoelectric crystallization | treatment | re-slurry | Residual rate |
| | (A − B)/A (%) | (B − C)/A (%) | (A − C)/A (%) | C/A (%) |
| Asp | 16.71% | −0.46% | 16.24% | 83.76% (collection |
| | | | | rate) |
| Glu | 97.67% | 0.17% | 97.85% | 2.15% |
| Ala | 98.40% | 0.72% | 99.12% | 0.88% |
| Val | 97.17% | 0.46% | 97.62% | 2.38% |
| Pyr | 100.00% | 0.00% | 100.00% | 0.00% |
| Mal | 90.21% | 8.56% | 98.76% | 1.24% |
| Ace | 100.00% | 0.00% | 100.00% | 0.00% |
| Suc | 93.78% | 6.22% | 100.00% | 0.00% |
| Fum | 99.88% | 0.12% | 100.00% | 0.00% |

| | | Concentrated | | | |
|---|---|---|---|---|---|
| | | solution | Filtrate (A) | | |
| | | (before activated | (sample to be | | Crystals |
| | Fermented | carbon | subjected to | | (C) |
| | clear liquid | treatment/suction | isoelectric | Crude | (final |
| | (S) | filtration) | crystallization) | crystals (B) | product) |
| Total Solids | 187.79 (g/L) | — | — | 99.76% | 100.00% |
| Asp total amount | 655.40 | 634.35 | 632.92 | 527.19 | 530.13 |
| (mmol) | | | | | |
| Asp concentration | 89.04 (g/L) | 307.80 (g/L) | 314.98 (g/L) | 0.78 (g/g) | 0.99 (g/g) |
| Asp purity (%)^{#1} | 47.42 | — | — | 78.19 | 99.00 |
| Asp collection | 100 | 96.79 | 96.57 | 80.44 | 80.89 |
| rate (%)^{#2} | | | | | |

^{#1}Percentage of value of each Asp concentration/each total solids
^{#2}Percentage of each Asp total amount (mmol)/Asp total amount (655.40 mmol) in fermented clear liquid (S)

As shown in FIGS. 2A to 2C, aspartic acid was held in a high proportion with respect to the total amount (632.92 mmol) of aspartic acid in the filtrate (A) subjected to the isoelectric crystallization through each purification step of the isoelectric crystallization and the thermal re-slurry treatment, and it was possible to produce aspartic acid crystals at a collection rate of 83.76% (that is, the loss was approximately 16%) in the end (Table 2 and FIG. 2C). On the other hand, a certain amount of a variety of amino acids other than aspartic acid that contaminated the filtrate (A) subjected to the isoelectric crystallization, that is, glutamic acid (57.57 mmol), alanine (187.21 mmol), and valine (32.81 mmol), were eliminated in a proportion of more than 97% with respect to the amount of each component in the filtrate (A) by the purification step of the isoelectric crystallization and, furthermore, could be each eliminated by the purification step of the thermal re-slurry treatment in proportions of 97.85%, 99.12% and 97.62%, respectively, in the end (Table 2 and FIGS. 3A to 3C).

Pyruvic acid (1.32 mmol), malic acid (76.07 mmol), acetic acid (90.9 mmol), succinic acid (18.34 mmol), and fumaric acid (8.19 mmol), which were a certain amount of a variety of organic acids that contaminated the filtrate (A), were effectively eliminated by the purification step of the isoelectric crystallization as described below. That is, 100% of pyruvic acid and acetic acid were eliminated through the purification step of the isoelectric crystallization, 99.88% of fumaric acid was also eliminated, and more than 90% of malic acid and succinic acid were also eliminated (Table 2 and FIG. 3A). In addition, regarding a small amount of fumaric acid, malic acid, and succinic acid that remained in the crude crystals (B) obtained through the isoelectric crystallization, 100% of three acids, that is, acetic acid, succinic acid, and fumaric acid were eliminated through the purification step of the additional thermal re-slurry treatment. While a small amount (0.94 mmol, residual rate: 1.24%) of malic acid remained, which means that, in other words, 98.76% of the total amount (76.07 mmol) contained in the filtrate (A) was eliminated, the purity of aspartic acid in the crystals (C), which were the final product, increased to 99.00%, and a high-purity aspartic acid crystal product was obtained (Table 2, FIGS. 2A to 2C, and FIGS. 3A to 3C).

Additionally, since white turbidity occurred when equal amounts of a 100 g/L solution of the aspartic acid crude crystals (B) and 100 mM barium chloride (manufactured by Wako Pure Chemical Industries, Ltd.) were mixed together, it was found that sulfuric acid ions were contained in the sample. In contrast, since white turbidity did not occur when equal amounts of a 100 g/L solution of the crystals (C) and 100 mM barium chloride (manufactured by Wako Pure Chemical Industries, Ltd.) were mixed together in the same manner, it was found that a considerable amount of sulfuric acid ions that remained in the crude crystals (B) immediately after the isoelectric point were also effectively eliminated through the thermal re-slurry treatment and the crystals (C), which were the final product, rarely contained sulfuric acid ions.

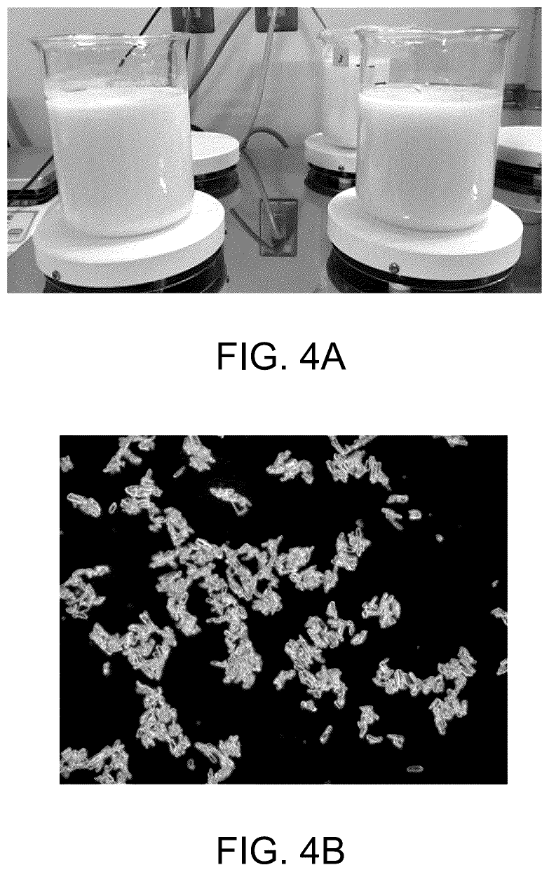

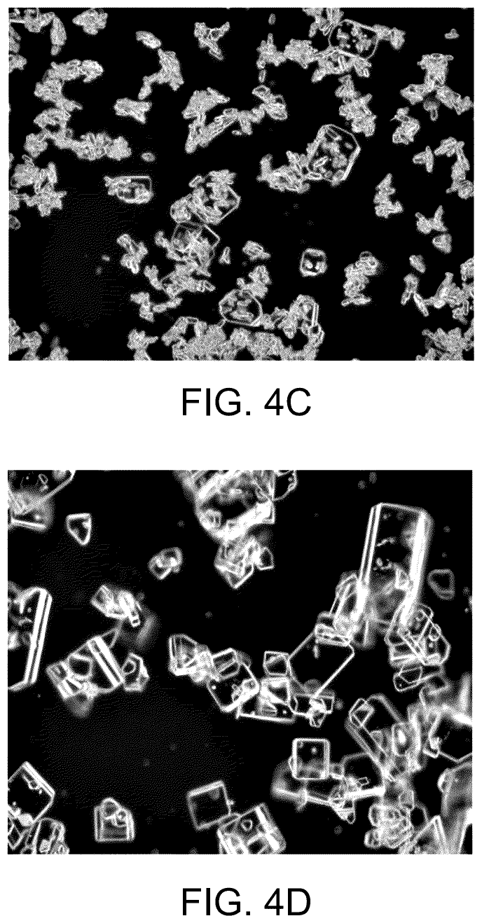

Furthermore, a photograph of each crude crystal (B) slurry sample captured in the process of performing the heating treatment (thermal re-slurry treatment) on the slurry sample of the crude crystals (B) is shown in FIG. 4A. A sample in the left beaker is the crude crystal (B) slurry sample before the heating treatment, and a sample in the right beaker is the crude crystal (B) slurry sample at a point in time where a predetermined time had elapsed after the heating treatment. As is clear from the photographs, both slurry samples have an appearance of a slurry having a predetermined turbidity, but the crude crystal (B) slurry sample before the heating treatment (the left beaker) has a white-turbid appearance whereas the crude crystal (B) slurry sample after the heating treatment (the right beaker) has a yellow/white-turbid appearance. This difference in appearance is attributed to the fact that the appearance turns from white turbid into yellow/white turbid as the time elapses after the heating treatment.

Furthermore, as a result of observing each crystal sample with a microscope (manufactured by Olympus Corporation, model No. CX41LF) at a predetermined point in time in the thermal re-slurry treatment, at a point in time where the sample temperature after the beginning of heating reached 70° C., fine columnar crude crystals were observed as shown in the photograph of FIG. 4B. Incidentally, after that, at a point in time where the sample temperature after the beginning of heating reached 77°C., relatively large plate-like crystals began to be generated from the columnar crude crystals as shown in the photograph of FIG. 4C, and, at a point in time where the sample temperature after the beginning of heating reached 100° C., almost all of the columnar crude crystals converted to plate-like crystals as shown in the photograph of FIG. 4D.

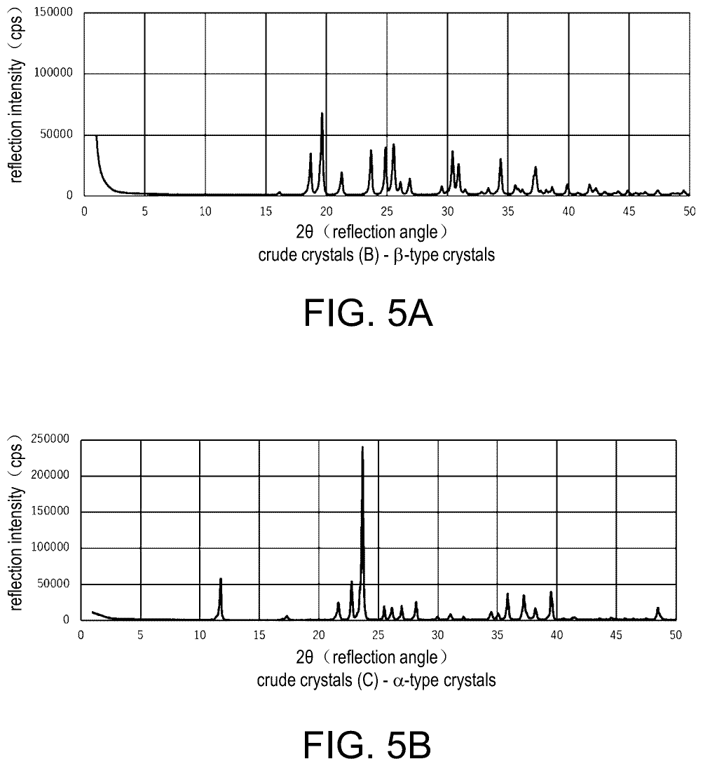

As the result of the above-described microscopic observation, it was assumed that aspartic acid was present in a β-type crystal form in the crude crystals (B) and the crystal form was converted to α-type by the subsequent heating treatment in the crystals (C), and the crude crystals (B) and the crystals (C) were analyzed by X-ray diffraction as described above. As a result, in an X-ray diffraction chart shown in FIG. 5A, a peak of diffracted X-ray was confirmed at each diffraction angle of 18.8°, 19.7°, and 25.0°, which confirmed that the crystal form of aspartic acid that crystallized in the crude crystals (B) was β-type crystals, and, in an X-ray diffraction chart shown in FIG. 5B, a peak of diffracted X-ray was confirmed at each diffraction angle of 21.65° and 23.7°, which confirmed that the crystal form of aspartic acid that crystallized in the crystals (C) was α-type crystals.

As described above, according to the embodiments of the present invention, it was suggested that, even in the case of using a crude product obtained by a fermentation method in which a microbe is used as a starting material, it is possible to hold a high collection rate of aspartic acid and efficiently eliminate other amino acids, organic acids, sulfuric acid ions, and the like, which correspond to impurities, and, furthermore, high-purity aspartic acid can be produced in a useful α-type crystal form in the end.

### [Test Example 2] Examination of Thermal Re-Slurry Temperature

Tests were performed by the same method as in Test Example 1 except that, in Test Example 1, the number of the test samples was changed to four and the test conditions for each test sample were changed as described below. That is, compared with the procedure in Test Example 1, in the present test example, the heating temperature in the section (4) “Heating treatment (thermal re-slurry method)” was changed to 70° C., 80° C., 90° C., and 100° C. for each sample. Additionally, the heating time employed for each sample is as described below in the following results.

### Results

As a result of microscopic observation in the purification step of the thermal re-slurry treatment, conversion from β-type crystals to α-type crystals was observed in all of the samples on which the heating treatment was performed at 70° C., 80° C., 90° C., and 100° C., respectively.

In more detail, in the sample on which the heating treatment was performed at 70° C., no crystal conversion was observed at a point in time where one hour elapsed from when the temperature of the sample reached 70° C. after the beginning of heating, but crystal conversion was admitted later in the middle of the sample being cooled to normal temperature. Furthermore, in the sample on which the heating treatment was performed at 80° C., the above-described crystal conversion was admitted at a point in time where approximately 16 minutes elapsed from when the temperature of the sample reached 80° C. Furthermore, in each of the samples on which the heating treatment was performed at 90° C. and 100° C., respectively, the above-described crystal conversion was admitted at a point in time where approximately 10 minutes elapsed from when the temperature of each sample reached the heating temperature of its own. As described above, according to the results of the samples heated at 70° C., 80° C., 90° C., and 100° C., respectively, crystal conversion was admitted in all of the samples, and it was confirmed that there is a tendency that, when the heating temperature is relatively high, the heating time necessary for crystal conversion to occur becomes short; on the other hand, as the heating temperature becomes lower, the heating time necessary for crystal conversion to occur becomes longer.

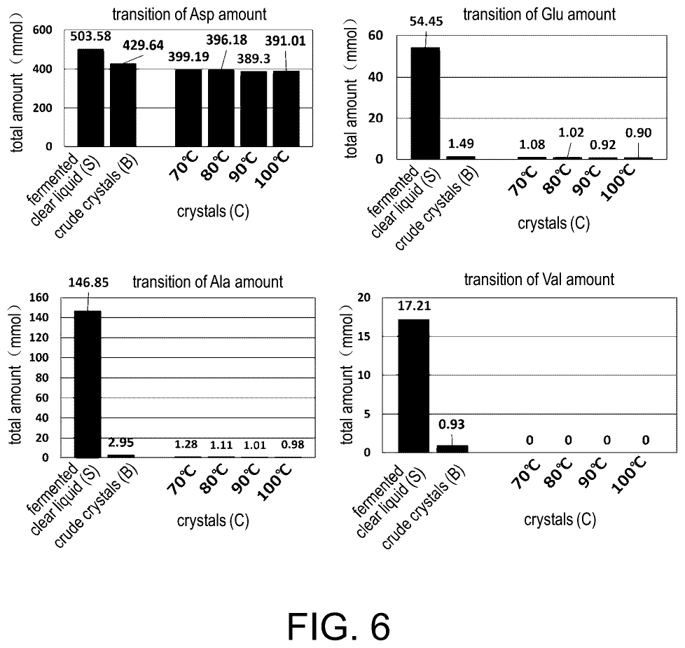

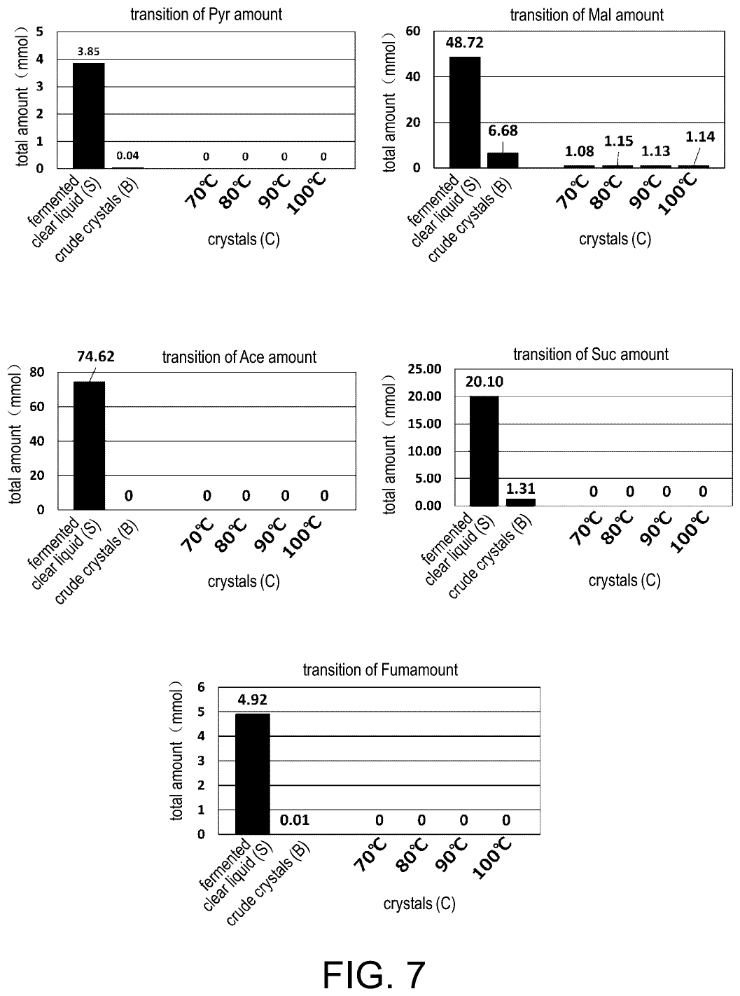

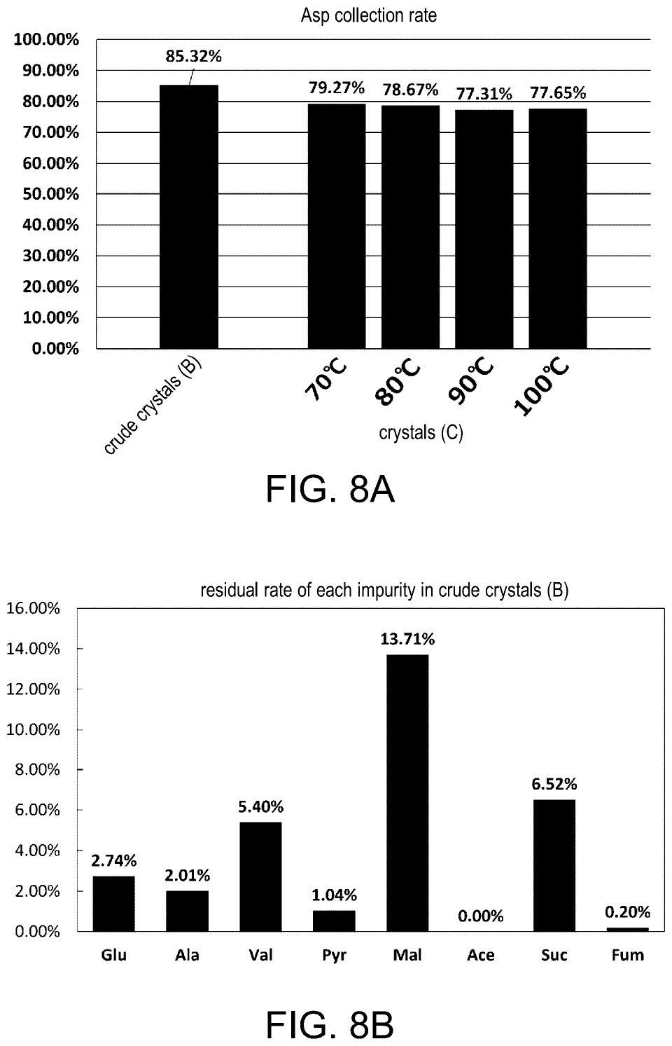

Next, the results of the amino acid analyses and the organic acid analyses are shown in Tables 3 and 4 and FIG. 6, FIG. 7, and FIG. 8A and 8B.

TABLE 3

| | |
|---|---|
| Fermented clear liquid | Crude crystals (B) |
| (S) | (after drying) |

| | | Total | | Total |
|---|---|---|---|---|
| | Concentration | amount | Concentration | amount |
| | (mmol/L) | (mmol) | (mmol/g) | (mmol) |
| Asp | 675.17 | 503.58 | 4.77 | 429.64 |
| Glu | 73.01 | 54.45 | 0.02 | 1.49 |
| Ala | 196.88 | 146.85 | 0.03 | 2.95 |
| Val | 23.08 | 17.21 | 0.01 | 0.93 |
| Pyr | 5.16 | 3.85 | 0.00 | 0.04 |
| Mal | 65.32 | 48.72 | 0.07 | 6.68 |
| Ace | 100.05 | 74.62 | 0.00 | 0.00 |
| Suc | 26.95 | 20.10 | 0.01 | 1.31 |
| Fum | 6.60 | 4.92 | 0.00 | 0.01 |

| | Crystals (C) | | Crystals (C) | | Crystals (C) | | Crystals (C) | |
|---|---|---|---|---|---|---|---|---|
| | heated at 70° C. | | heated at 80° C. | | heated at 90° C. | | heated at 100° C. | |
| | | Total | | Total | | Total | | Total |
| | Concentration | amount | Concentration | amount | Concentration | amount | Concentration | amount |
| | (mmol/g) | (mmol) | (mmol/g) | (mmol) | (mmol/g) | (mmol) | (mmol/g) | (mmol) |
| Asp | 7.33 | 399.19 | 7.31 | 396.18 | 7.19 | 389.30 | 7.21 | 391.01 |
| Glu | 0.02 | 1.08 | 0.02 | 1.02 | 0.02 | 0.92 | 0.02 | 0.90 |
| Ala | 0.02 | 1.28 | 0.02 | 1.11 | 0.02 | 1.01 | 0.02 | 0.98 |
| Val | 0.00 | 0.00 | 0.00 | 0.00 | 0.00 | 0.00 | 0.00 | 0.00 |
| Pyr | 0.00 | 0.00 | 0.00 | 0.00 | 0.00 | 0.00 | 0.00 | 0.00 |
| Mal | 0.02 | 1.08 | 0.02 | 1.15 | 0.02 | 1.13 | 0.02 | 1.14 |
| Ace | 0.00 | 0.00 | 0.00 | 0.00 | 0.00 | 0.00 | 0.00 | 0.00 |
| Suc | 0.00 | 0.00 | 0.00 | 0.00 | 0.00 | 0.00 | 0.00 | 0.00 |
| Fum | 0.00 | 0.00 | 0.00 | 0.00 | 0.00 | 0.00 | 0.00 | 0.00 |

TABLE 4

| | Crude | | | | |
|---|---|---|---|---|---|
| | crystals (B) | Crystals (C) residual rate (C/S) | | | |
| | residual rate | Heated | Heated | Heated | Heated |
| | (B/S) | at 70° C. | at 80° C. | at 90° C. | at 100° C. |
| Asp | 85.32% | 79.27% | 78.67% | 77.31% | 77.65% |
| Glu | 2.74% | 1.98% | 1.87% | 1.69% | 1.65% |
| Ala | 2.01% | 0.87% | 0.76% | 0.69% | 0.67% |
| Val | 5.40% | 0.00% | 0.00% | 0.00% | 0.00% |
| Pyr | 1.04% | 0.00% | 0.00% | 0.00% | 0.00% |
| Mal | 13.71% | 2.22% | 2.36% | 2.32% | 2.34% |
| Ace | 0.00% | 0.00% | 0.00% | 0.00% | 0.00% |
| Suc | 6.52% | 0.00% | 0.00% | 0.00% | 0.00% |
| Fum | 0.20% | 0.00% | 0.00% | 0.00% | 0.00% |

* Residual rate relative to total amount of individual components in fermented clear liquid (S)

As shown in Table 3 and FIG. 6, the amount of aspartic acid transited at a high level through the fermented clear liquid (S), the crude crystals (B), and the crystals (C) with no significant losses, a collection rate of 85.32% was exhibited in the crude crystals (B), and it was possible to collect aspartic acid in the crystals (C) within a range of 77% to 80% in the end (Table 4 and FIG. 8A).

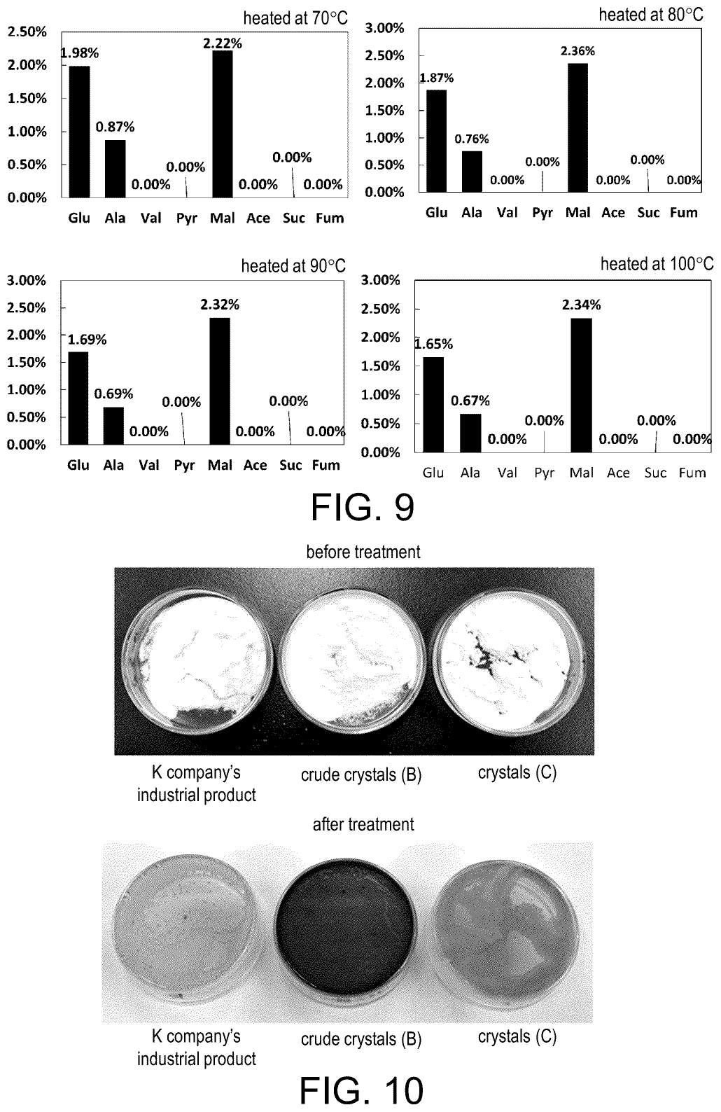

On the other hand, as can be read from Table 3 and FIG. 6, a considerable amount of a variety of amino acids other than aspartic acid, that is, glutamic acid, alanine, and valine contaminated the fermented clear liquid (S) while the amount was small compared with the total amount of aspartic acid, but a considerable amount thereof was eliminated through the isoelectric crystallization, and the residual rate in the crude crystals (B) was reduced to less than 6% (Table 4 and FIG. 8B). Furthermore, the residual rate of these amino acids other than aspartic acid was reduced to less than 2% in the crystals (C), and, particularly, valine was eliminated 100% in all of the samples regardless of the heating treatment temperature, and the residual rate exhibited 0% (Table 4 and FIG. 9).

Furthermore, as can be read from Table 3 and FIG. 7, a considerable amount of pyruvic acid, malic acid, acetic acid, succinic acid, fumaric acid, where were the kinds of organic acid, contaminated the fermented clear liquid (S), but a considerable amount thereof was eliminated through the isoelectric crystallization. In more detail, in the crude crystals (B), the residual rate of malic acid can be said to be relatively high, malic acid was eliminated to 13.71%, pyruvic acid and succinic acid were reduced to less than 10%, furthermore, fumaric acid was reduced to 0.20%, and acetic acid was completely eliminated (Table 4 and FIG. 8B).

Furthermore, as a point to which attention needs to be paid, in all of the crystals (C) obtained by the heating treatment at 70° C., 80° C., 90° C., and 100° C. as well, all of acetic acid, succinic acid, and fumaric acid were completely eliminated through the thermal re-slurry treatment, the residual rate of malic acid was approximately 2.2% to 2.4% while the remaining of malic acid was confirmed, and it was confirmed that each sample according to the crystals (C) had a high quality suitable for a purified product of aspartic acid (Table 4 and FIG. 9).

As described above, in Test Example 2, it was indicated that the α-type crystals of aspartic acid were collected at a high collection rate, and, in particular, it was indicated that, when the crude crystals were treated at a relatively high temperature in the purification step of the thermal re-slurry treatment, it was possible to generate α-type crystals of aspartic acid within a short time and to effectively eliminate amino acids other than aspartic acid and a variety of organic acids.

### Evaluation Test: Polymerization and Coloration Test

A polymerization coloring test was performed as described below using 2 g of each of the β-type crude crystals (after the isoelectric crystallization) and the α-type crystals obtained by performing the heating treatment (thermal re-slurry method) at 100° C. that were obtained in Test Example 2 and, as a contrast, a commercially available aspartic acid powder (manufactured by Kyowa Hakko Bio Co., Ltd., high-purity grade).

2 g of each of the above-described samples and 2 mL of 85% phosphoric acid (manufactured by FUJIFILM Wako Pure Chemical Corporation) were mixed together on a 60 mm glass Petri dish, this mixture was injected into a constant temperature dryer (manufactured by AS ONE Corporation, model No. OFW-300B) and heated at 160° C. for 16 hours to 20 hours to polymerize aspartic acid. As a result of visually observing the sample after the heating treatment, it was confirmed that, while strong brown coloration was confirmed in the sample of the β-type crude crystals after the isoelectric crystallization, coloration was suppressed in the α-type crystal sample obtained by the thermal re-slurry treatment at 100° C., and the α-type crystal sample has a comparable quality with the high purity-grade aspartic acid powder, which was the contrast as shown in FIG. 10A and 10B.

### Test Example 3

(1) Concentration/activated carbon treatment

5 L of a fermented clear liquid of the modified Corynebacterium glutamicum was vacuum-concentrated in the same manner as in the method described in the section (1) of Test Example 1, and a concentrated solution in which the aspartic acid concentration was estimated to be 2.5 M was obtained. Next, 4 g of powdered activated carbon (“CARBORAFFIN” manufactured by Osaka Gas Chemicals Co., Ltd.) per 100 g of aspartic acid was added to the obtained concentrated solution and stirred at normal temperature for 60 minutes, and then a solution was separated into activated carbon and a filtrate (A) by a suction filtration method.

### (2) Isoelectric Crystallization

350 mL of the filtrate (A) was poured into each of three beakers, and they were subjected to isoelectric crystallization as a no-seed crystal-added sample (i), a seed crystal-added sample (ii), and a seed crystal-added sample (iii), respectively. Specifically, sulfuric acid was slowly added to each of the samples under stirring at normal temperature, and the pH of each sample solution was adjusted to near 2.77, which is the isoelectric point of aspartic acid, using a pH meter (“D-71” manufactured by Horiba, Ltd.), thereby crystallizing aspartic acid in each sample. The sulfuric acid added to the no-seed crystal-added sample (i), the seed crystal-added sample (ii), and the seed crystal-added sample (iii) were 86.00 g, 86.69 g and 88.90 g, respectively. Additionally, to the samples (ii) and (iii) to which seed crystals were added, approximately 0.1 g of aspartic acid crude crystals were added when the pH of each solution passed 5.5. Furthermore, at the time of the isoelectric crystallization, since the temperature of each sample solution increased up to approximately 70° C., the sample solution was left to stand under stirring until normal temperature was reached, then, the stirring was stopped, and the sample solution was cooled to 4° C.

As a result of performing isoelectric crystallization as described above on each solution according to each of the samples (i) to (iii), each solution exhibited a transparent amber color before the isoelectric crystallization, but turned into a solution of a light-yellow white turbid color after the isoelectric crystallization, and the crystallization of crude crystals in the solution was visually observed. FIG. 11 shows the photographs of the appearance of the sample (i) before the isoelectric crystallization (left side) and after the isoelectric crystallization (right side), respectively.

Each sample in which the crude crystals were generated was separated into a supernatant and crude crystals (solid matter) by performing a suction filtration method thereon. 450 mL of ultrapure water was poured over each crude crystal sample obtained as described above to eliminate impurities attached to the crystal surfaces. Each wet crude crystal sample after the washing was moved to a stainless steel square tray, injected into the constant temperature dryer (manufactured by AS ONE Corporation, model No. OFW-300B) and dried at 55° C. Furthermore, each dried crude crystal sample was crushed using a mixer (“BKE-07” manufactured by Hanwa Co., Ltd.) and each collected in a plastic container.

116.17 g of crude crystals (B1) derived from the no-seed crystal-added sample (i), 122.31 g of crude crystals (B2) derived from the seed crystal-added sample (ii), and 116.24 g of a crude crystal sample (B3) derived from the seed crystal-added sample (iii) were obtained as described above.

A part of each of the crude crystals (B1), (B2), and (B3) was subjected to an amino acid analysis and an organic acid analysis in the same manner as in Test Example 1.

### (3) Heating Treatment (Thermal Re-Slurry Treatment)

100 g of each of the crude crystals (B1), (B2), and (B3) was weighed with an electronic balance (manufactured by Shimadzu Corporation, model No. UW6200H). Each of the measured crude crystal samples was suspended in ultrapure water so that the final volume reached 334 mL, thereby preparing a 30% crude crystal slurry. This 30% crude crystal slurry was stirred and heated in a beaker using a hot stirrer (manufactured by AS ONE Corporation, model No. HS-360H). The heating was stopped after 10 minutes from when the temperature of each sample reached 100° C., and then the sample was left to stand until normal temperature was reached under stirring. After that, the stirring was stopped, and each sample was cooled at 4° C.

Next, each sample was separated into a supernatant and a crystalline fraction (solid matter) by performing the suction filtration method thereon. 100 mL of ultrapure water was poured over each crystal sample obtained as described above to eliminate impurities attached to the crystal surfaces. A wet crystal sample after the washing was moved to a stainless steel square tray, injected into the constant temperature dryer (manufactured by AS ONE Corporation, model No. OFW-300B) and dried at 55° C. all night. Next, each crystal sample was made into a powder using a dispensing spoon and collected in a plastic container.

93.05 g of crystals (C1) derived from the no-seed crystal-added sample (i), 88.98 g of crystals (C2) derived from the seed crystal-added sample (ii), and 93.22 g of a crystal sample (C3) derived from the seed crystal-added sample (iii) were obtained as described above. A part of each of the crystal samples (C1), (C2), and (C3) was subjected to an amino acid analysis and an organic acid analysis in the same manner as in Test Example 1.

### Results

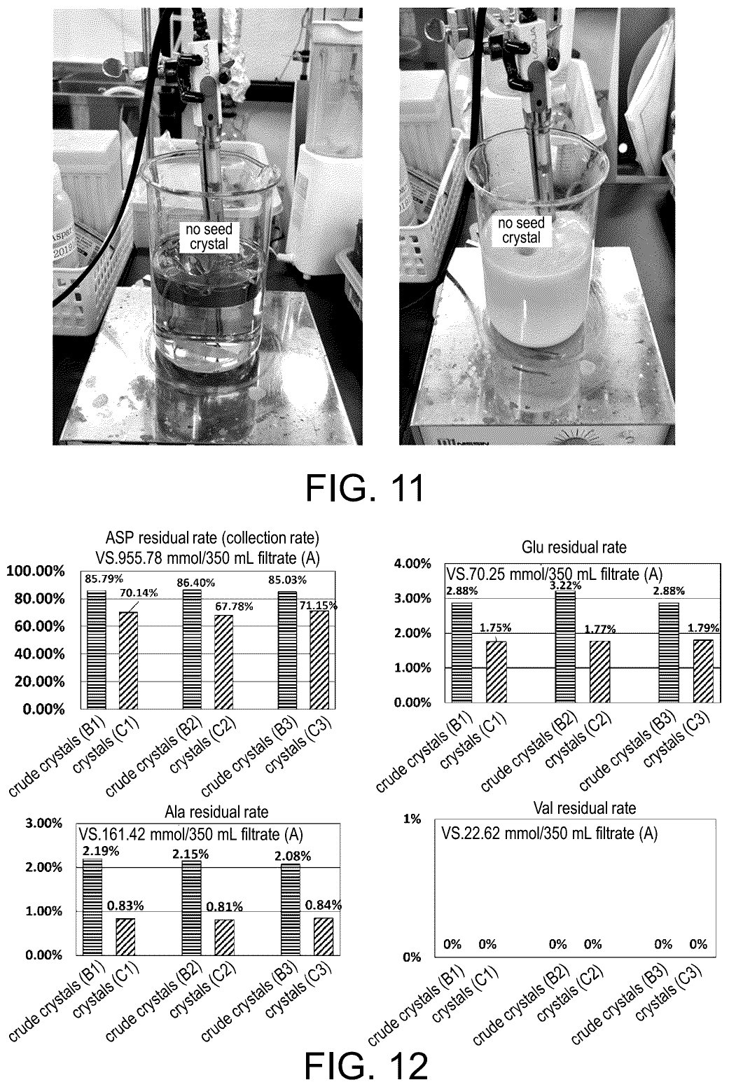

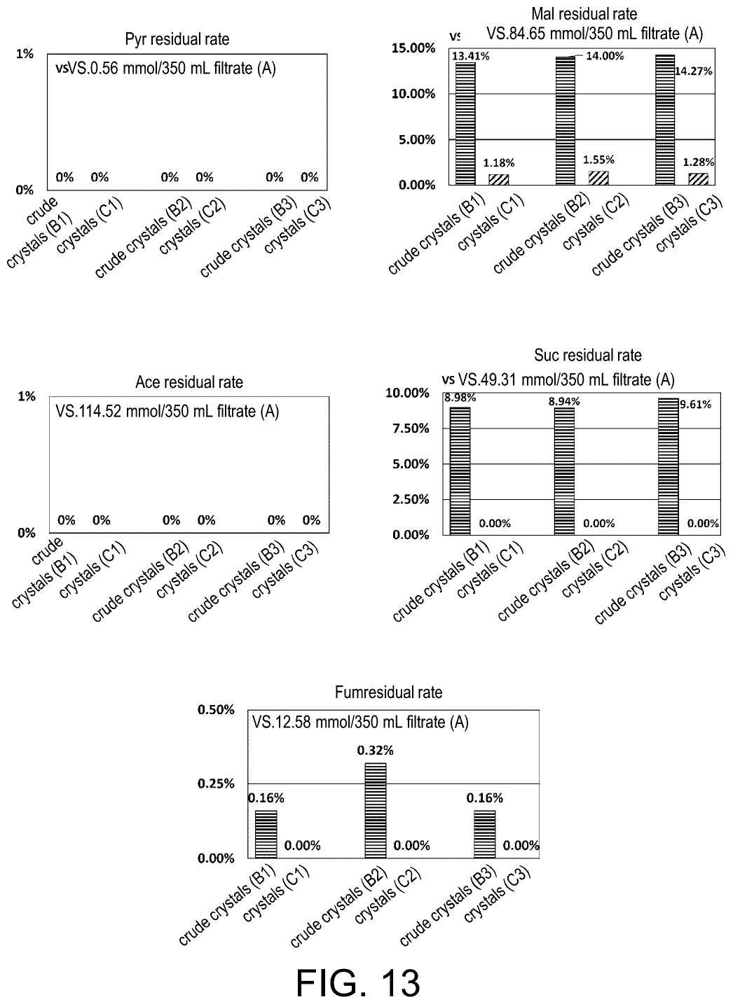

The results of a variety of amino acid analyses and organic acid analyses are shown in Tables 5 to 7 and FIGS. 12 and 13. In detail, Table 5 shows the concentrations and total amounts of a variety of components of the fermented clear liquid (S), the concentrated solution of the fermented clear liquid (S) obtained by the vacuum concentration treatment, the filtrate (A) after the activated carbon treatment and the suction filtration, the crude crystals (B1) to (B3) obtained by the isoelectric treatment and the crystals (C1) to (C3) obtained by the thermal re-slurry treatment. Furthermore, Table 6 and FIGS. 12 and 13 show the residual rates of a variety of components in each of the crude crystals (B1) to (B3) and the crystals (C1) to (C3). The residual rate corresponds to a value of the proportion (%) of the total amount of each component in each crude crystal sample or crystal sample relative to the total amount of each component in the filtrate (A) subjected to the isoelectric crystallization. Additionally, Table 7 shows the total amount, purity (%) and collection rate (%) of Asp in each sample.

TABLE 5

| | | | Concentrated | | | | | | | | | |
|---|---|---|---|---|---|---|---|---|---|---|---|---|
| | | | solution | | | | | | | | | |
| | | | (before activated | | | | | | | | | |
| | | | carbon | | | | | | | | | |
| | Fermented clear | | treatment/suction | | Filtrate (A) | | Crude crystals | | Crude crystals | | Crude crystals | |
| | liquid (S) | | filtration) | | (350 mL) | | (B1) | | (B2) | | (B3) | |
| | Concen- | Total | Concen- | Total | Concen- | Total | Concen- | Total | Concen- | Total | Concen- | Total |
| | tration | amount | tration | amount | tration | amount | tration | amount | tration | amount | tration | amount |
| | (mmol/L) | (mmol) | (mmol/L) | (mmol) | (mmol/L) | (mmol) | (mmol/g) | (mmol) | (mmol/g) | (mmol) | (mmol/g) | (mmol) |
| Asp | 567.44 | 2837.20 | 2587.87 | 2872.53 | 2730.81 | 955.78 | 7.06 | 819.99 | 6.75 | 825.76 | 6.99 | 812.69 |
| Glu | 41.61 | 208.06 | 190.45 | 211.40 | 200.71 | 70.25 | 0.02 | 2.02 | 0.02 | 2.26 | 0.02 | 2.02 |
| Ala | 96.11 | 480.55 | 441.93 | 490.54 | 461.20 | 161.42 | 0.03 | 3.54 | 0.03 | 3.47 | 0.03 | 3.35 |
| Val | 12.07 | 60.33 | 62.81 | 69.72 | 64.64 | 22.62 | 0.00 | 0.00 | 0.00 | 0.00 | 0.00 | 0.00 |
| Pyr | 1.02 | 5.11 | 1.60 | 1.77 | 1.60 | 0.56 | 0.00 | 0.00 | 0.00 | 0.00 | 0.00 | 0.00 |
| Mal | 53.24 | 266.18 | 240.89 | 267.39 | 241.86 | 84.65 | 0.10 | 11.35 | 0.10 | 11.85 | 0.10 | 12.08 |
| Ace | 81.26 | 406.32 | 343.65 | 381.45 | 327.19 | 114.52 | 0.00 | 0.00 | 0.00 | 0.00 | 0.00 | 0.00 |
| Suc | 42.57 | 212.83 | 194.58 | 215.98 | 140.88 | 49.31 | 0.04 | 4.43 | 0.04 | 4.41 | 0.04 | 4.74 |
| Fum | 7.66 | 38.29 | 34.14 | 37.90 | 35.95 | 12.58 | 0.00 | 0.02 | 0.00 | 0.04 | 0.00 | 0.02 |

| | Crystals (C1) | | Crystals (C2) | | Crystals (C3) | |
|---|---|---|---|---|---|---|
| | Concentration | Total amount | Concentration | Total amount | Concentration | Total amount |
| | (mmol/g) | (mmol) | (mmol/g) | (mmol) | (mmol/g) | (mmol) |
| Asp | 7.20 | 670.35 | 7.28 | 647.78 | 7.30 | 680.08 |
| Glu | 0.01 | 1.23 | 0.01 | 1.24 | 0.01 | 1.26 |
| Ala | 0.01 | 1.34 | 0.01 | 1.30 | 0.01 | 1.36 |
| Val | 0.00 | 0.00 | 0.00 | 0.00 | 0.00 | 0.00 |
| Pyr | 0.00 | 0.00 | 0.00 | 0.00 | 0.00 | 0.00 |
| Mal | 0.01 | 1.00 | 0.01 | 1.31 | 0.01 | 1.08 |
| Ace | 0.00 | 0.00 | 0.00 | 0.00 | 0.00 | 0.00 |
| Suc | 0.00 | 0.00 | 0.00 | 0.00 | 0.00 | 0.00 |
| Fum | 0.00 | 0.00 | 0.00 | 0.00 | 0.00 | 0.00 |

*The crude crystals (B) and the crystals (C) were measured by adjusting an analysis sample to 1 g/10 mL and subjecting the sample to HPLC analysis.

TABLE 6

| | Residual rate | | | Residual rate | | |
|---|---|---|---|---|---|---|
| | (crude crystals B/filtrate A) | | | (crystals C/filtrate A) | | |
| | Crude crystals | Crude crystals | Crude crystals | Crystals | Crystals | Crystals |
| | (B1) | (B2) | (B3) | (C1) | (C2) | (C3) |
| Asp | 85.79% | 86.40% | 85.03% | 70.14% | 67.78% | 71.15% |
| Glu | 2.88% | 3.22% | 2.88% | 1.75% | 1.77% | 1.79% |
| Ala | 2.19% | 2.15% | 2.08% | 0.83% | 0.81% | 0.84% |
| Val | 0.00% | 0.00% | 0.00% | 0.00% | 0.00% | 0.00% |
| Pyr | 0.00% | 0.00% | 0.00% | 0.00% | 0.00% | 0.00% |
| Mal | 13.41% | 14.00% | 14.27% | 1.18% | 1.55% | 1.28% |
| Ace | 0.00% | 0.00% | 0.00% | 0.00% | 0.00% | 0.00% |
| Suc | 8.98% | 8.94% | 9.61% | 0.00% | 0.00% | 0.00% |
| Fum | 0.16% | 0.32% | 0.16% | 0.00% | 0.00% | 0.00% |

TABLE 7

| | Fermented | | | | | |
|---|---|---|---|---|---|---|
| | clear liquid | Concentrated | Filtrate (A) | Crude | Crude | Crude |
| | (S) | solution | (350 mL) | crystals (B1) | crystals (B2) | crystals (B3) |
| Total Solids | — | 734.206 (g/L) | — | 99.67% | 99.65% | 99.73% |
| Asp total | 2837.20 | 2872.53 | 955.78 | 819.99 | 825.76 | 812.69 |
| amount | | | | | | |
| (mmol) | | | | | | |
| Asp | 75.53 (g/L) | 344.45 (g/L) | 363.47 (g/L) | 0.94 (g/g) | 0.90 (g/g) | 0.93 (g/g) |
| concentration | | | | | | |
| Asp purity | — | 46.91% | — | 94.31% | 90.32% | 93.25% |
| (%)^{#1} | | | | | | |
| Asp | — | | — | 85.79% | 86.40% | 85.03% |
| collection | | | | | | |
| rate (%)^{#2} | | | | | | |

| | Crystals (C1) | Crystals (C2) | Crystals (C3) |
|---|---|---|---|
| Total Solids | 100.01% | 99.99% | 100.00% |
| Asp total amount (mmol) | 670.35 | 647.78 | 680.08 |
| Asp concentration | 0.96 (g/g) | 0.97(g/g) | 0.97(g/g) |
| Asp purity (%)^{#1} | 95.99% | 97.01% | 97.00% |
| Asp collection rate (%)^{#2} | 70.14% | 67.77% | 71.15% |

^{#1}Percentage of value of each Asp concentration/each total solids
^{#2}Each Asp total amount (mmol)/Asp total amount (955.78 mmol) per 350 mL of filtrate (A)

As shown in Table 6 and FIG. 12, aspartic acid was held in a high proportion with respect to the total amount (955.78 mmol) of aspartic acid in the filtrate (A) subjected to the isoelectric crystallization through each purification step of the isoelectric crystallization and the thermal re-slurry treatment, and it was possible to produce aspartic acid crystals at a collection rate of approximately 67% to 71% in the crystals (C1) to (C3) in the end. On the other hand, a certain amount of a variety of amino acids other than aspartic acid that contaminated the filtrate (A) subjected to the isoelectric crystallization were subjected to the purification step of the isoelectric crystallization, whereby glutamic acid (70.25 mmol) was reduced to a residual rate of approximately 3%, alanine (161.42 mmol) was reduced to a residual rate of approximately 2%, furthermore, valine (22.62 mmol) was completely eliminated even in all of the crude crystals (B1) to (B3) (Table 6 and FIG. 12). Furthermore, a small amount of glutamic acid and alanine that remained in the crude crystals (B1) to (B3) were subjected to the purification step of the thermal re-slurry treatment and were thereby reduced to residual rates of approximately 1.8% and approximately 0.8%, respectively, in the crystals (C1) to (C3) (FIG. 12).

Furthermore, a certain amount of a variety of organic acids that contaminated the filtrate (A), that is, pyruvic acid (0.56 mmol), malic acid (84.65 mmol), acetic acid (114.52 mmol), succinic acid (49.31 mmol), and fumaric acid (12.58 mmol), were also effectively eliminated as shown in Table 6 and FIG. 13. That is, 100% of pyruvic acid and acetic acid were eliminated through the purification step of the isoelectric crystallization (FIG. 13). Furthermore, a certain amount of succinic acid and fumaric acid remained in the crude crystals (B), but were completely eliminated in the crystals (C1) to (C3) (FIG. 13). Additionally, a small amount of malic acid remained through the crude crystals (B) and the crystals (C1) to (C3), but the residual rate thereof in the crystals (C1) to (C3), which were final products, was below 2% (FIG. 13), the purity of aspartic acid exhibited high values of approximately 96% to 97% in all of the crystal samples, and high-purity aspartic acid α-type crystals were obtained (Table 7).

As one of the points to which attention needs to be paid in the present test example, it was indicated that, according to one embodiment of the present invention, it is possible to effectively eliminate a variety of amino acids or organic acids other than aspartic acid that contaminated crude products obtained by the fermentation method while purifying a desired crystal form of aspartic acid even in the sample (i) to which no seed crystals were added as in the samples (ii) and (iii) to which the seed crystals were added.

### [Test Example 4] Examination of Isoelectric Crystallization Temperature

Tests were performed by the same method as in (1) to (6) of Test Example 1 except that, in Test Example 1, the test was performed by maintaining and controlling the temperature of the solution in the section (2) “Isoelectric crystallization” to each of 30° C., 50° C., and 80° C. using a water bath or an oil bath.

### Results

As a result of analyzing each sample of the crude crystals (B) prepared by performing the isoelectric crystallization at a temperature maintained and controlled to each of 30° C., 50° C., and 80° C., it was confirmed that all samples contained aspartic acid in a high proportion. Furthermore, it was admitted that all of the samples contained a variety of amino acids other than aspartic acid, that is, glutamic acid, alanine, and valine and contained organic acids other than aspartic acid. In an X-ray diffraction chart of each sample according to the crude crystals (B), regardless of the treatment temperature, the samples exhibited a peak of diffracted X-ray at each diffraction angle of 18.8°, 19.7°, and 25.0° as in the chart shown in FIG. 5A. Therefore, it was admitted that the crystal form of aspartic acid that crystallized in each sample according to the crude crystals (B) was all β-type crystals.

As a result of performing a heating treatment (thermal re-slurry treatment) according to the method described in (4) of Test Example 1 on each sample according to the crude crystals (B) prepared as described above, similar to Test Example 1, the conversion of the crystal shape was admitted in all samples. That is, when the appearance of the slurry sample before the heating treatment was observed with a microscope, fine columnar crystals were observed; however, after the heating treatment, at the latest at a point in time where the sample temperature after the beginning of heating reached 100° C., it was confirmed that almost all of the columnar crystals converted to plate-like crystals.

Furthermore, as a result of analyzing each sample according to each of the crystals (C) obtained by separating crystals after the heating treatment, it was confirmed that, in all of the samples, the aspartic acid purity was 99.00% or higher and high-purity aspartic acid crystals could be obtained. In addition, it was confirmed that all of the samples according to the crystals (C), which were the final product through the heating treatment, rarely contained sulfuric acid ions. Additionally, when each sample was analyzed by X-ray diffraction, in the X-ray diffraction charts of all of the samples, similar to the chart shown in FIG. 5B, a peak of diffracted X-ray was admitted at each diffraction angle of 21.65° and 23.7°. Therefore, it was found that the crystal form of aspartic acid in each sample according to the crystals (C) was all α-type crystals.

As described above, it was confirmed that, even in a case where the step of the isoelectric crystallization treatment was performed at a temperature controlled within a wide temperature range of 30° C., 50° C., and 80° C., aspartic acid in a β-type crystal form was obtained. In summary, it was indicated that the isoelectric crystallization treatment step that is performed within such a wide temperature range can be combined with the subsequent thermal re-slurry treatment step, and, according to one embodiment of the present invention, it is possible to produce high-purity aspartic acid in a useful α-type crystal form in the end even in the case of using a crude product obtained by a fermentation method in which a microbe is used as a starting material.

### [Test Example 5] Examination of Thermal Re-Slurry Temperature

### (1) Concentration/Activated Carbon Treatment

Concentration/activated carbon treatment were performed by the same method as in Test Example 1 except that, as the starting material that was subjected to the concentration treatment, 3 L of a fermented clear liquid of the Asp-producing Corynebacterium used in Test Example 1 was used and the activated carbon treatment was performed by stirring a sample to which powdered activated carbon was added for 60 minutes or longer while maintaining the temperature at 60° C. The amount of the obtained concentrated solution (filtrate (A)) was 720 mL.

### (2) Isoelectric Crystallization

First, the concentrated solution obtained as described above was heated to 50° C. using a microbe bioreactor (manufactured by ABLE Corporation & Biott Corporation, model No. BMJ-01NC). After that, the heater was turned off, 55 g of sulfuric acid was added to the sample under stirring for 50 minutes, and the pH was adjusted to near 2.73 so that the pH of the cooled sample at normal temperature reached near 2.77, thereby crystallizing aspartic acid. At the time of this pH adjustment, 0.05 g of aspartic acid crude crystals were added to the sample as seed crystals at a point in time where the pH reached 5.0 and 4.7, respectively. Since the temperature of the sample increased up to approximately 60° C. at the time of adding the seed crystals to the sample, the sample was left to stand under stirring until normal temperature was reached, then, crystals were taken out, put into a beaker, and cooled at 4° C.

A variety of analyses were performed on the sample in each stage in the same manner as in Test Example 1, and the contamination with dihydroxyacetone (DHA), which can become a colorant, was also analyzed and evaluated by a normal method of HPLC in addition to a variety of amino acid/organic acid analyses.

Test conditions other than what has been described above are the same as in Test Example 1.

### (3) Crystal Separation

A crude crystal sample was obtained under the same conditions as in Test Example 1 except that, in the operation for crystal separation in Test Example 1, 400 mL of ultrapure water was poured over a crystal product obtained by the above-described isoelectric crystallization five times to wash crystals and eliminate impurities attached to the surfaces of the crystals. The amount of the obtained crude crystal sample was 87.91 g.

### (4) Heating Treatment (Thermal Re-Slurry Treatment)

From the crude crystal sample obtained in the section (3), 30.0 g of dried crude crystals were weighed with an electronic balance (manufactured by Shimadzu Corporation, model No. UW6200H), and 70 g of ultrapure water was added thereto, thereby preparing 30% (w/w %) crude crystal slurry samples. While these crude crystal slurry samples were each maintained at temperatures of 40° C., 45° C., 60° C., and 70° C. and stirred using a water bath (manufactured by TAITEC Corporation, model No. SM-05N) and a stirrer (manufactured by Nissin Rika Co., Ltd., model No. SW-501J), crystal conversion in each sample was observed using a microscope (manufactured by Olympus Corporation, model No. CX41LF). Regarding the heating time, at a point in time where crystal conversion was admitted in the sample after the beginning of heating, the heating was stopped, immediately after the crystal conversion could be confirmed, the samples were left to stand under stirring until normal temperature was reached, then, the stirring was stopped, and each sample was cooled at 4° C.

Each sample was cooled and then separated into solid and liquid by a suction filtration method, and 30 mL of ultrapure water was poured over each of the obtained solid samples to wash crystals and eliminate impurities attached to the surfaces. Wet crystals after the washing were moved to an aluminum steel square tray and dried at 55° C. using a constant temperature dryer (manufactured by AS ONE Corporation, model No. OFW-300B). Next, the crystals were crushed with a dispensing spoon and collected in a plastic container, each sample was subjected to an amino acid/organic acid analysis, and the residual rate of impurities was calculated.

### Results

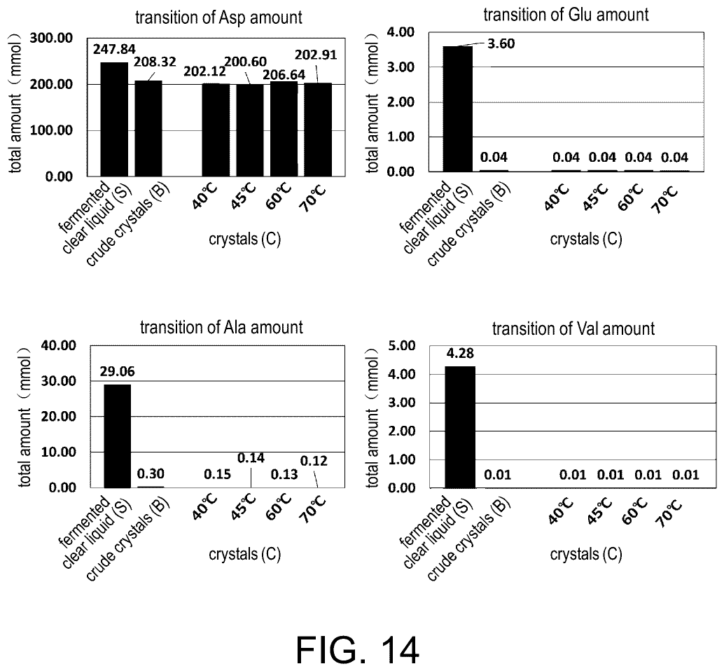

The analysis results of amino acids, a variety of organic acids, and dihydroxyacetone (DHA) are shown in Tables 8 to 10, FIGS. 14, 15, 16A and 16B, and FIG. 17.

TABLE 8

| | |
|---|---|
| | Crude crystals (B) |
| Fermented clear liquid (S) | (after drying) |

| | Concentration | Total amount | Concentration | Total amount |
|---|---|---|---|---|
| | (mmol/L) | (mmol) | (mmol/L) | (mmol) |
| Asp | 606.51 | 247.84 | 6.94 | 208.32 |
| Glu | 8.81 | 3.60 | 0.00 | 0.04 |
| Ala | 71.11 | 29.06 | 0.01 | 0.30 |
| Val | 10.48 | 4.28 | 0.00 | 0.01 |
| Pyr | 2.33 | 0.95 | 0.00 | 0.00 |
| DHA | 1.80 | 0.73 | 0.00 | 0.00 |
| Mal | 29.20 | 11.93 | 0.08 | 2.48 |
| Ace | 61.94 | 25.31 | 0.00 | 0.00 |
| Suc | 13.10 | 5.35 | 0.00 | 0.00 |
| Fum | 3.23 | 1.32 | 0.00 | 0.00 |

| | Crystals (C) | | Crystals (C) | | Crystals (C) | | Crystals (C) | |
|---|---|---|---|---|---|---|---|---|
| | heated at 40° C. | | heated at 45° C. | | heated at 60° C. | | heated at 70° C. | |
| | | Total | | Total | | Total | | Total |
| | Concentration | amount | Concentration | amount | Concentration | amount | Concentration | amount |
| | (mmol/L) | (mmol) | (mmol/L) | (mmol) | (mmol/L) | (mmol) | (mmol/L) | (mmol) |
| Asp | 7.09 | 202.12 | 7.07 | 200.60 | 7.18 | 206.64 | 7.18 | 202.91 |
| Glu | 0.00 | 0.04 | 0.00 | 0.04 | 0.00 | 0.04 | 0.00 | 0.04 |
| Ala | 0.01 | 0.15 | 0.00 | 0.14 | 0.00 | 0.13 | 0.00 | 0.12 |
| Val | 0.00 | 0.01 | 0.00 | 0.01 | 0.00 | 0.01 | 0.00 | 0.01 |
| Pyr | 0.00 | 0.00 | 0.00 | 0.00 | 0.00 | 0.00 | 0.00 | 0.00 |
| DHA | 0.00 | 0.00 | 0.00 | 0.00 | 0.00 | 0.00 | 0.00 | 0.00 |
| Mal | 0.01 | 0.17 | 0.00 | 0.12 | 0.01 | 0.15 | 0.00 | 0.08 |
| Ace | 0.00 | 0.00 | 0.00 | 0.00 | 0.00 | 0.00 | 0.00 | 0.00 |
| Suc | 0.00 | 0.00 | 0.00 | 0.00 | 0.00 | 0.00 | 0.00 | 0.00 |
| Fum | 0.00 | 0.00 | 0.00 | 0.00 | 0.00 | 0.00 | 0.00 | 0.00 |

TABLE 9

| | Crude crystals | | | | |
|---|---|---|---|---|---|
| | (B) | Crystals (C) residual rate (C/S) | | | |
| | residual rate | Heated | Heated | Heated | Heated |
| | (B/S) | at 40° C. | at 45° C. | at 60° C. | at 70° C. |
| Asp | 84.06% | 81.55% | 80.94% | 83.37% | 81.87% |
| Glu | 1.23% | 1.11% | 1.09% | 1.10% | 1.06% |
| Ala | 1.03% | 0.52% | 0.49% | 0.44% | 0.41% |
| Val | 0.25% | 0.24% | 0.23% | 0.22% | 0.21% |
| Pyr | 0.00% | 0.00% | 0.00% | 0.00% | 0.00% |
| DHA | 0.00% | 0.00% | 0.00% | 0.00% | 0.00% |
| Mal | 20.78% | 1.44% | 0.97% | 1.25% | 0.65% |
| Ace | 0.00% | 0.00% | 0.00% | 0.00% | 0.00% |
| Suc | 0.00% | 0.00% | 0.00% | 0.00% | 0.00% |
| Fum | 0.00% | 0.00% | 0.00% | 0.00% | 0.00% |

* Residual rate relative to total amount of individual components in fermented clear liquid (S)

TABLE 10

| | Thermal re-slurry treatment temperature | | | |
|---|---|---|---|---|
| | Heated | | | |
| | at 40° C. | 45° C. | 60° C. | 70° C. |
| Asp amount (g) in crude crystals | 27.73 | 27.73 | 27.73 | 27.73 |
| (B) | | | | |
| Asp amount (g) in crystals (C) | 26.91 | 26.70 | 27.51 | 27.01 |
| Asp collection rate | 97.04% | 96.29% | 99.21% | 97.40% |

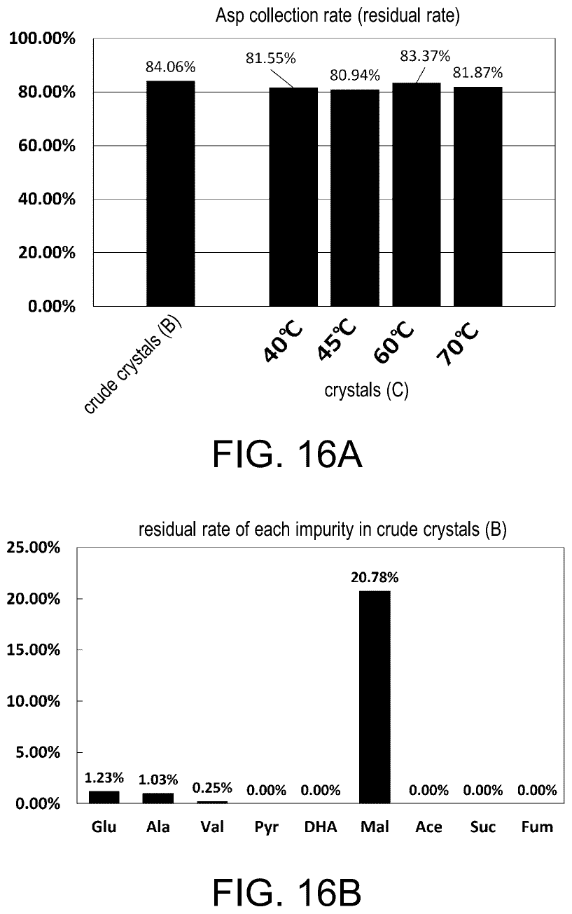

In all of the samples for which 40° C., 45° C., 60° C., and 70° C. were each employed as the thermal re-slurry treatment temperature, the amount of aspartic acid transited at a high level through the fermented clear liquid (S), the crude crystals (B), and the crystals (C) with no significant losses (Table 8 and FIG. 14), a residual rate of 84.06% was exhibited in the crude crystals (B), and an aspartic acid residual rate of approximately 80% was exhibited in the crystals (C) in the end (Table 9 and FIG. 16A). Furthermore, as is found from Table 10, the collection rates of aspartic acid in the crystals (C) with respect to the crude crystals (B) show that, in all of the samples for which the above-described thermal re-slurry treatment temperatures were employed, it was possible to attain high collection rates of approximately 95%.

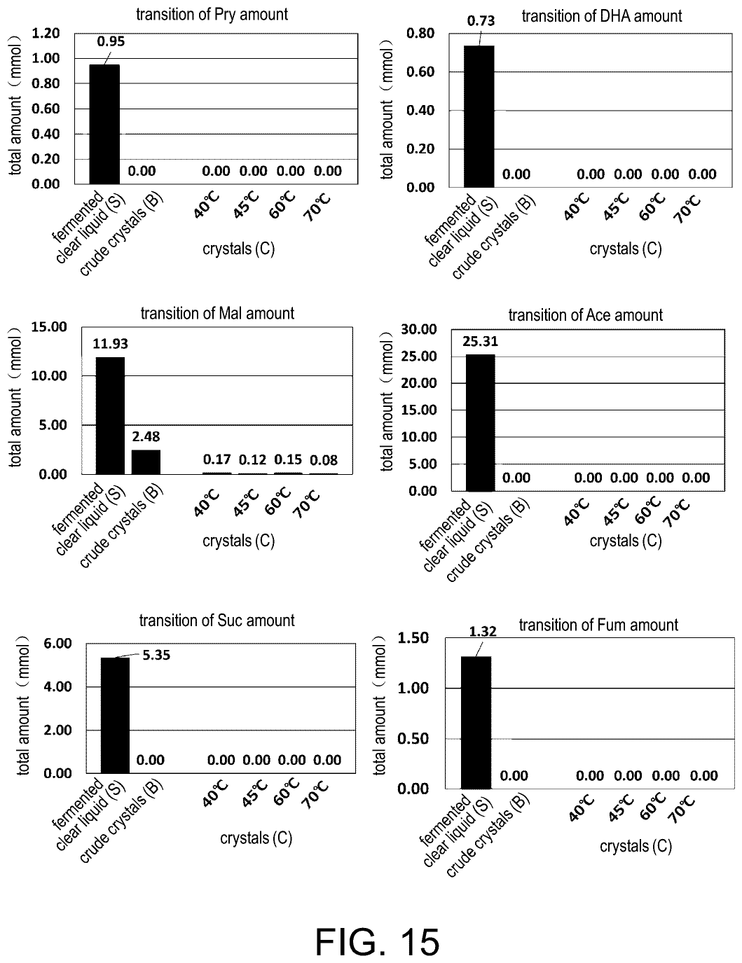

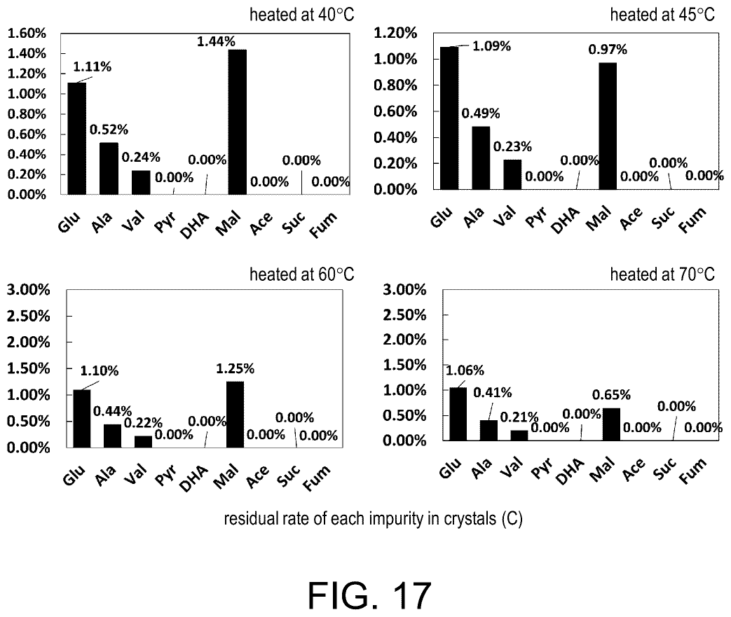

Incidentally, a considerable amount, while small compared with the total amount of aspartic acid, of a variety of amino acids other than aspartic acid, a variety of organic acids, and dihydroxyacetone (DHA) contaminated the fermented clear liquid (S), but it was found that, similar to Test Example 2, a considerable amount of each contaminant was removed through each step of the isoelectric crystallization and the subsequent thermal re-slurry (Table 8, FIG. 14, and FIG. 15). In detail, regarding a variety of amino acids other than aspartic acid, it was found that, at a point in time of the crude crystals (B), glutamic acid and alanine exhibited a residual rate of approximately 1%, valine exhibited a residual rate of slightly 0.25%, and almost all of glutamic acid, alanine, and valine were eliminated by the step of the isoelectric crystallization, and, in all of the crystals (C) samples for which each thermal re-slurry treatment temperature was employed, an additional decrease in each residual rate was admitted (Table 9 and FIG. 14). Furthermore, the residual rates of a variety of organic acids in each sample of the crude crystals (B) show that, while a considerable amount, which was approximately 20%, of malic acid contaminated the sample, each of the other organic acids exhibited a value of 0% and was not detected (Table 9 and FIG. 16B). Furthermore, it was admitted that, in each sample of the crystals (C), which were the product, malic acid was reduced to approximately 1% through the thermal re-slurry step in which each of the above-described treatment temperatures was employed (Table 9 and FIG. 17). Furthermore, regarding dihydroxyacetone (DHA), which becomes a causative substance of coloration, it was found that the fermented clear liquid (S) was confirmed to be contaminated with dihydroxyacetone (Table 8 and FIG. 15), but a value of 0% was exhibited at a point in time of the crude crystals (B) obtained through the isoelectric crystallization, and dihydroxyacetone was eliminated to a high purity through the isoelectric crystallization (Tables 8 and 9, FIG. 15, and FIG. 16).

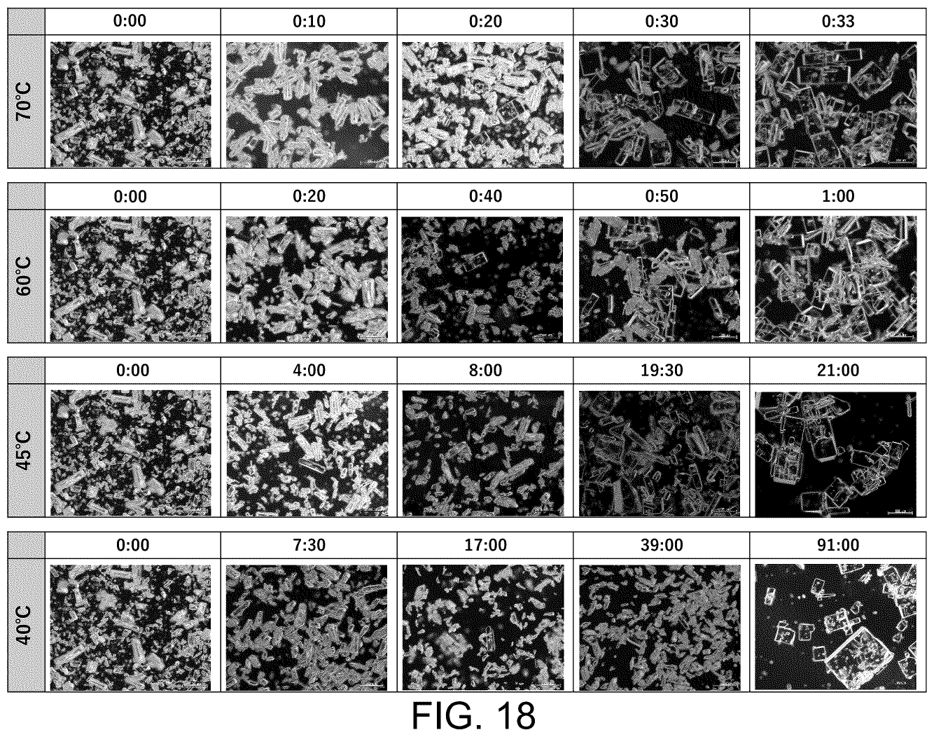

Next, the results of observing each sample for which the thermal re-slurry treatment temperatures (40° C., 45° C., 60° C., and 70° C.) were each employed between the thermal re-slurry treatments and over time with a microscope are shown in FIG. 18. The numbers shown over the micrographs at each thermal re-slurry treatment temperature indicate the elapsed times from the beginning of heating, and the unit thereof is “hours” [hours (h): minutes (min)].

In all of the samples of the crystals (C) obtained by employing each of the thermal re-slurry treatment temperatures, fine columnar crystals were admitted at the extremely initial phase of the thermal re-slurry treatment; however, when the heating treatment was begun, a situation in which the columnar crystals (beta-type crystals) were converting to plate-like crystals (α-type crystals) as the time elapsed was observed. In detail, in the samples for which 60° C. and 70° C., which can be said to be relatively high temperatures as the thermal re-slurry treatment temperature, were each employed, crystal particles almost completely converted plate-like crystals (α-type crystals) at points in time where approximately one hour and approximately 30 minutes elapsed, respectively. Furthermore, even in each sample for which 45° C. and 40° C., which can be said to be relatively high temperatures as the thermal re-slurry treatment temperature, were employed, crystal particles almost completely converted plate-like crystals (α-type crystals) at points in time where approximately 21 hours and approximately 91 hours elapsed, respectively.

In the present test example as well, similar to Test Example 1, the crystal form was analyzed by X-ray diffraction regarding each sample according to the crude crystals (B) and the crystals (C). As a result, regarding each sample according to the crude crystals (B), a peak of diffracted X-ray was confirmed at each diffraction angle of 18.8°, 19.7°, and 25.0° as in the chart shown in FIG. 5A, and it was thus found that the crystal form of aspartic acid that crystallized in each sample was all β-type crystals.

Furthermore, regarding each sample according to the crystals (C), a peak of diffracted X-ray was admitted at each diffraction angle of 21.65° and 23.7° as in the chart shown in FIG. 5B, and it was thus found that the crystal form of aspartic acid in each sample was all α-type crystals.

According to the present test example, it was suggested that, even in a case where the thermal re-slurry treatment was performed not only within a high temperature range of 70° C. or higher but also within a relatively low temperature range including 40° C., 45° C., 60° C., and the like, it is possible to convert beta-type crystals to α-type crystals by setting a considerable heating treatment time and the collection of the α-type crystals at a high collection rate and a high purity becomes possible through a predetermined step of the present invention.

As described above, according to the embodiments of the present invention, it was shown with favorable reproducibility that, even in the case of using a crude product obtained by a fermentation method in which a microbe is used as a starting material, regardless of whether seed crystals are added or not, it is possible to maintain a high collection rate of aspartic acid and efficiently eliminate other amino acids, organic acids, sulfuric acid ions, and the like, which correspond to impurities, and, furthermore, aspartic acid can be produced in a useful α-type crystal form in the end.

### INDUSTRIAL APPLICABILITY

Since aspartic acid can be used as a raw material in producing chemical materials, for example, food, cosmetics, medicines, water-absorbing/biodegradable amino acid polymers, the present invention is highly industrially applicable.

What is claimed is:

1. A method for producing aspartic acid, comprising:
(q) preparing a slurry of a crystalline fraction (X) containing β-type crystals of aspartic acid and at least one impurity; and
(r) heating the slurry to convert the β-type crystals of aspartic acid to α-type crystals and then obtain a crystalline fraction (Y) containing aspartic acid in the α-type crystals.

2. The method according to claim 1,
wherein, in the step (r), the slurry is heated in a temperature range of 30° C. to 190° C. to convert the β-type crystals of aspartic acid to α-type crystals.

3. The method according to claim 1,
wherein, in the step (r), the slurry is heated in a temperature range of 60° C. to 150° C. to convert the β-type crystals of aspartic acid to α-type crystals.

4. The method according to claim 1, further comprising:
(p) in a solution (S) containing aspartic acid or a salt thereof and at least one impurity, adjusting a pH of the solution (S) to a predetermined pH value in an acidic region to generate β-type crystals of aspartic acid and then separate a fraction containing the β-type crystals from the solution (S),
wherein, in the step (q), a slurry of the crystalline fraction (X) is prepared using the fraction containing the β-type crystals.

5. The method according to claim 4,
wherein, in the step (p), the pH of the solution (S) is adjusted to a predetermined value within a range of 0.50 to 6.95 to generate the β-type crystals of aspartic acid.

6. The method according to claim 4,
wherein, in the step (p), the pH of the solution (S) is adjusted to a predetermined value within a range of 1.50 to 4.50 to generate the β-type crystals of aspartic acid.

7. The method according to claim 4,
wherein the solution (S) that is subjected to the step (p) contains a seed crystal.

8. The method according to claim 7,
wherein the seed crystal contains β-type crystals of aspartic acid.

9. The method according to claim 4,
wherein the solution (S) that is subjected to the step (p) is a culture obtained by culturing or reacting a microbe in a culture medium, a clear liquid separated from the culture or a concentrate thereof.

10. The method according to claim 4,
wherein the solution (S) that is subjected to the step (p) is a solution containing the aspartic acid or a salt thereof at a concentration of 0.1 to 5.0 M.

11. The method according to claim 4,
wherein the solution (S) that is subjected to the step (p) contains, as the impurity, at least one selected from the group consisting of amino acids other than aspartic acid, organic acids, and salts thereof.

12. The method according to claim 4,
wherein the solution (S) that is subjected to the step (p) contains, as the impurity, at least
i) at least one selected from the group consisting of glutamic acid, alanine, valine, and a salt thereof, and
ii) at least one selected from the group consisting of pyruvic acid, malic acid, acetic acid, succinic acid, fumaric acid, and a salt thereof.

13. The method according to claim 4,
wherein the pH of the solution (S) that is subjected to the step (p) is within a range of 6.00 to 8.00.

14. The method according to claim 13,
wherein, in the step (p), an acid is added to the solution (S) to adjust the pH of the solution (S) to a predetermined value within a range of 1.00 to 6.85 and generate the β-type crystals of aspartic acid.

15. The method according to claim 4,
wherein, in the step (p), after the β-type crystals of aspartic acid are generated in the solution (S), the fraction containing the β-type crystals is separated from the solution (S) by a solid-liquid separation method,
in the step (q), the slurry of the crystalline fraction (X) is prepared using the fraction containing the β-type crystals separated in the step (p), and,
in the step (r), the slurry is heated, the β-type crystals of aspartic acid are converted to α-type crystals, and then the crystalline fraction (Y) is separated from the slurry of the crystalline fraction (X) by the solid-liquid separation method.

16. The method according to claim 15,
wherein, in the step (p), after a crystalline fraction containing the β-type crystals is separated from the solution (S) by the solid-liquid separation method, the separated crystalline fraction is washed with a solvent once or more and, furthermore, dried,
in the step (q), the slurry of the crystalline fraction (X) is prepared using the dried crystalline fraction, and,
in the step (r), after the crystalline fraction (Y) is separated from the slurry of the crystalline fraction (X) by the solid-liquid separation method, the separated crystalline fraction (Y) is washed with a solvent once or more and, furthermore, dried.
